(19) United States
(12) Patent Application Publication
(10) Pub. No.: US 2015/0110716 A1
(43) Pub. Date: Apr. 23, 2015

(54) METHODS OF USING SPECT/CT ANALYSIS FOR STAGING CANCER

(71) Applicant: Molecular Insight Pharmaceuticals, Inc., Tarrytown, NY (US)

(72) Inventor: Thomas ARMOR, Hooksett, NH (US)

(73) Assignee: Molecular Insight Pharmaceuticals, Inc., Tarrytown, NY (US)

(21) Appl. No.: 14/517,760

(22) Filed: Oct. 17, 2014

Related U.S. Application Data
(60) Provisional application No. 61/892,931, filed on Oct. 18, 2013.
(60) Provisional application No. 61/932,212, filed on Jan. 27, 2014.
(60) Provisional application No. 61/932,686, filed on Jan. 28, 2014.
(60) Provisional application No. 61/954,183, filed on Mar. 17, 2014.
(60) Provisional application No. 61/955,095, filed on Mar. 18, 2014.
(60) Provisional application No. 62/007,747, filed on Jun. 4, 2014.
(60) Provisional application No. 62/064,962, filed on Oct. 16, 2014.

(51) Int. Cl. A61K 51/04

(52) U.S. Cl. CPC A61K 51/0453
USPC 424/1.65

(57) ABSTRACT

A method of evaluating a subject suspected of harboring a prostrate tumor includes administering to the subject an effective amount of a gamma-emitting transition metal complex conjugated to a targeting moiety that selectively binds to prostate-specific membrane antigen (PSMA), including PSMA expressed on the surface of a prostate tumor; subjecting the subject to a nuclear medicine tomographic imaging technique to obtain one or more images of at least a portion of prostate tissue that comprises tumor lesions; assessing a level of uptake of said gamma-emitting transition metal complex conjugated to a targeting moiety by said at least a portion of prostate tissue compared to a level of uptake by control tissue; and determining if a ratio of the level of uptake by said at least a portion of prostate tissue compared the level of uptake by control tissue is at or above a predetermined threshold.

### CROSS-REFERENCE TO RELATED APPLICATIONS

This application claims the benefit of U.S. Provisional Patent Application Nos. 61/892,931, filed on Oct. 18, 2013; 61/932,212, filed on Jan. 27, 2014, 61/932,686, filed on Jan. 28, 2014; 61/954,183, filed on Mar. 17, 2014; 61/955,095, filed on Mar. 18, 2014; 62/007,747, filed on Jun. 4, 2014; and 62/064,962, filed on Oct. 16, 2014, the entire disclosures of which are incorporated herein by reference for any and all purposes.

### FIELD

The present technology is generally related to the imaging of prostate cancer (PCa) tissue to differentiate cancerous tissue from normal tissue or benign prostate tissue. Specifically, the present technology relies on determining the ratio of the uptake of a radiolabeled compound that selectively binds to prostate specific membrane antigen (PSMA), which is overexpressed on the surface of prostate cancer tumors to the uptake of the same compound by a control tissue to differentiate clinically significant disease from silent or indolent disease within the prostate. Thus, compounds according to the present technology permit the detection of primary and metastatic prostate cancer tumors.

### BACKGROUND

Radiopharmaceuticals may be used as diagnostic or therapeutic agents by virtue of the physical properties of their constituent radionuclides. Thus, their utility is not based on any pharmacologic action per se. Most clinical drugs of this class are diagnostic agents incorporating a gamma-emitting nuclide that, because of physical, metabolic or biochemical properties of its coordinated ligands, localizes in a specific organ after intravenous injection. The resultant images may reflect organ structure or function. These images are obtained by means of a gamma camera that detects the distribution of ionizing radiation emitted by the radioactive molecules.

In radioimaging, the radiolabel is a gamma-radiation emitting radionuclide that may be imaged using a gamma-radiation detecting camera (this process is often referred to as gamma scintigraphy). The imaged site is detectable because the radiotracer is chosen either to localize at a pathological site (termed positive contrast) or, alternatively, the radiotracer is chosen specifically not to localize at such pathological sites (termed negative contrast).

It is known that tumors may express unique proteins associated with their malignant phenotype or they may over-express normal constituent proteins in greater number than normal cells. The expression of distinct proteins on the surface of tumor cells offers the opportunity to diagnose and characterize disease by probing the phenotypic identity and biochemical composition of such a tumor protein. Radioactive molecules that selectively bind to specific tumor cell surface proteins allow the use of noninvasive imaging techniques for detecting the presence and quantity of tumor associated proteins, thereby providing vital information related to the diagnosis and extent of disease progression. In addition, radiopharmaceuticals can not only be used to image disease, but they may also be used to deliver a therapeutic radionuclide to the diseased tissue. The expression of peptide receptors and other ligand receptors on tumors makes them attractive targets to exploit for noninvasive imaging as well as targeted radiotherapy.

A critical challenge in imaging prostate cancer (PCa) is to differentiate clinically significant disease from silent or indolent disease within the prostate, as well as the identification of metastatic and recurrent disease. Imaging of PCa lesions within the prostate is challenging with computed tomography (CT) or magnetic resonance imaging (MRI) techniques. The protein prostate specific membrane antigen (PSMA) is up-regulated in cancer cells. Thus, a PSMA targeted radiotracer would be an ideal imaging agent for diagnosis of prostate cancer and to evaluate the extent of disease progression in a subject harboring prostate cancer.

A variety of radionuclides are known to be useful for radioimaging, including Ga-67, Tc-99m, In-111, I-123, and I-131. Perhaps the most widely used radioisotope for medical imaging is Tc-99m. Its 140 keV gamma-photon is ideal for use with widely-available gamma cameras. It has a short (6 hour) half-life, which is desirable when considering patient dosimetry. Finally, Tc-99m is readily available at relatively low cost through commercially-produced ⁹⁹Mo/Tc-99m generator systems.

### SUMMARY

In one aspect, Tc-99m labeled PSMA targeting radioimaging agents are provided for the differentiation of cancerous tissue from normal or benign tissue and for the evaluation of the progress of disease in a prostate cancer patient. In another aspect, a method of evaluating a human subject suspected of harboring a prostrate tumor is provided. According to such methods, an effective amount of a gamma-emitting transition metal complex conjugated to a targeting moiety that selectively binds to prostate-specific membrane antigen (PSMA), including PSMA expressed on the surface of a prostate tumor is administered to the subject. Following administration, the subject is imaged using a nuclear medicine tomographic imaging technique. One or more images of at least a portion of prostate tissue having tumor lesions are obtained. From these images the level of uptake of the gamma-emitting transition metal complex conjugated to a targeting moiety by at least a portion of prostate tissue is compared to a level of uptake by control tissue is assessed. In accordance with the method, the assessment is carried out by determining if the ratio of the level of uptake by at least a portion of prostate tissue to the level of uptake by a control tissue is below, at, or above a predetermined threshold value.

In one embodiment, the predetermined threshold is 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9 or 7.0 and is chosen statistically to minimize undesirable effects of false positives and false negatives. In one embodiment, the predetermined threshold has a value of 5.9. The method also permits evaluation of a subject harboring a prostate tumor to be conducted non-invasively. Imaging of the subject following administration of the gamma-emitting transition metal complex conjugated to a targeting moiety can be performed using any nuclear medicine tomographic imaging technique that is suitable for detecting gamma radiation. Illustrative imaging techniques include without limitation two-dimensional planar imaging, single-photon emission computed tomography (SPECT), and single-photon emission computed tomography combined with conventional computed tomography (SPECT/CT).

The control tissue that is used for determining the ratio of the uptake level can be any normal tissue, for example, normal pelvic muscle tissue or non-tumorous portions of prostate tissue. As mentioned above, the method provides a physician the necessary information to evaluate whether or not the subject has prostate cancer and whether the subject needs to undergo active surveillance or watchful-waiting or needs to undergo surgery, for instance radical prostatectomy, cryosurgery, radiation therapy, hormone (or androgen deprivation) therapy, chemotherapy, PSMA antibody-drug-conjugate, or combinations thereof if it is determined that the ratio is at or above 5.9. The phrase “active surveillance” and the phrase “watchful-waiting” are art recognized terms. See, for example American Cancer Society (2012) Review incorporated by reference herein in its entirety.

In one embodiment of the method, a subject may not be elected to undergo radical prostatectomy, cryosurgery, radiation therapy, hormone (or androgen deprivation) therapy, chemotherapy, PSMA antibody-drug-conjugate, or combinations thereof if it is determined that the ratio is below 5.9. According to another embodiment, the human subject undergoes active surveillance monitoring if it is determined that the ratio below 5.9. Under such circumstances the human subject is reevaluated periodically using the PSMA targeting radioimaging agents described herein.

According to another embodiment, the human subject undergoes watchful-waiting if it is determined that the ratio below 5.9. Under such circumstances the human subjects' symptoms are monitored.

The method may be used to detect tumor lesions in tissues other than prostate tissue. According to one embodiment, the radioimaging agent used is a Formula 1 compound. The compound represented by Formula (1) is a glutamic acid-urea-glutamic acid dimer to which a radionuclide chelating group is bonded via a linker. The transition metal radionuclide used for imaging is technetium-99m.

According to the method, the human subject is harboring a prostate cancer tumor if it is determined that the ratio is at or above 5.9. The method further suggests that the human patient harbors a prostate cancer tumor that would garner a Gleason score of about 7.0 or above, such as a high grade prostate cancer if it is determined that the ratio falls in the range of about 5.9 to about 13.0. According to another aspect, the method suggests that the human patient harbors a prostate cancer tumor that would garner a Gleason score of about 9.0 or above, if it is determined that the ratio falls in the range of about 15.5 to about 45.0. A ratio below 5.9 suggests a no disease state, that is, that the human subject does not harbor a prostate cancer.

In another aspect, a non-surgical method of identifying a severity level of prostate cancer in a patient harboring biopsy-confirmed prostate cancer is provided. The method includes administering to the patient an effective amount of a compound that is ^{99m}Tc-trofolastat chloride; determining a level of uptake of the compound in the prostate of the patient as a tumor (T) level; determining a level of uptake of the compound in a control tissue as a baseline (B) level; and assigning a severity level in terms of Gleason score if a ratio of T:B is at, or above, a predetermined threshold value. In some embodiments, the threshold value of >5.9 corresponds to a Gleason score of about 7.0 or greater. In some embodiments, the threshold value of about 15.5 or greater corresponds to a Gleason score of about 9.0 or greater. In some embodiments, the patient has not received a prior prostate cancer treatment. In some embodiments, the determining comprises obtaining an image of the patient using nuclear medicine tomographic imaging techniques.

In another aspect, a method is provided for confirming tumor metastasis to a pelvic lymph node of a prostate cancer patient. In one embodiment, a compound represented by Formula 1 or Formula 2 which selectively binds to prostate-specific membrane antigen (PSMA), is administered to a prostate cancer patient. Following administration of the compound represented by Formula 1 or Formula 2, the pelvis of the patient is imaged to obtain one or more images and the level of uptake of the compound by at least a portion of a pelvic lymph node of the prostate cancer patient is assessed by comparing to a level of uptake by control tissue.

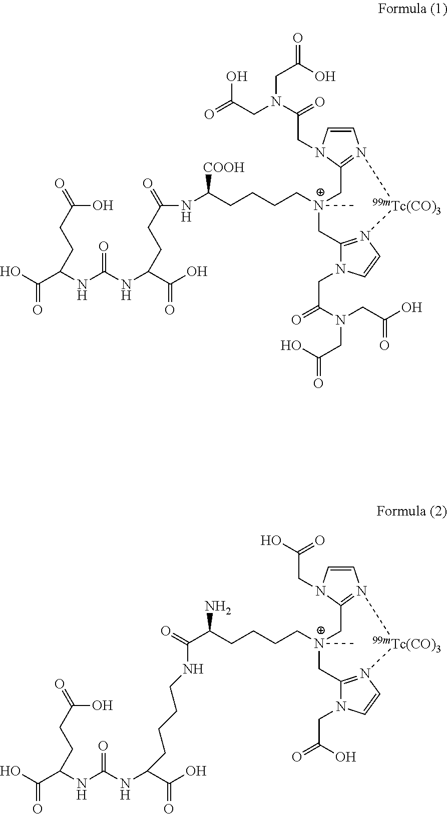

According to the method, metastasis of a tumor is confirmed if it is determined that a ratio of the level of uptake of the compound by at least a portion of a pelvic lymph node to the level of uptake by control tissue is at, or above, a predetermined threshold value. In some embodiments, the predetermined value as it related to metastasis is at least about 30. In some embodiments the predetermined value is about 30. According to an aspect of the method, the patient is administered an effective amount of a compound of Formula (1).

Imaging of the human subject after administration may be performed using a nuclear medicine tomographic imaging technique such as two-dimensional planar imaging, single-photon emission computed tomography (SPECT), or single-photon emission computed tomography combined with conventional computed tomography (SPECT/CT). A patient with confirmed pelvic lymph node metastasis may further be subjected to surgery, for example, radical prostatectomy, cryosurgery, radiation therapy, hormone (or androgen deprivation) therapy, chemotherapy, PSMA antibody-drug-conjugate, or combinations thereof. The control tissue may be selected from normal prostate tissue, normal pelvic muscle, or normal pelvic lymph node. See American Cancer Society (2012) Review, which is incorporated herein by reference.

In another embodiment, a method for monitoring a status of prostate cancer in a human subject is provided. According to the method, a subject with prostate cancer is administered an effective amount of a gamma-emitting imaging agent comprising a prostate specific-membrane antigen (PSMA) recognition moiety and a radionuclide. Following such administration, the subject is imaged by a nuclear medicine tomographic imaging technique to obtain one or more images of at least a portion of prostate tissue that includes tumor lesions. The level of uptake of the gamma-emitting transition metal complex conjugated to a targeting moiety by the portion of prostate tissue is then compared a level of uptake by control tissue to facilitate the determination of a ratio based on the level of uptake by a prostate tissue to the level of uptake by control tissue. This ratio is compared to a baseline ratio previously determined for the human subject to monitor the status of prostate cancer.

The imaging agent used may be a glu-urea-glu or glu-urea-lys based compound, such as a compound represented by Formula (1) or Formula (2) or a pharmaceutically acceptable salt thereof. In one aspect of this method the imaging step is carried out 1-4 hours after the administering step. According to the method, a ratio that is above the baseline ratio suggests worsening of the prostate cancer condition in a subject and a ratio below the baseline ratio suggests that the prostate cancer condition has not worsened.

In another aspect, a method is provided for confirming tumor metastasis in a prostate cancer patient. The method includes administering to the patient an effective amount of a compound that selectively binds to prostate-specific membrane antigen (PSMA), the compound represented by Formula 1 or Formula 2 or a pharmaceutically acceptable salt thereof; imaging a region of interest in the subject; obtaining a level of uptake of the compound by the prostate of the prostate cancer patient as a target (T) level; obtaining a level of uptake of the compound in control tissue (B); obtaining a quantitative score as a ratio of T:B; and confirming metastasis if it is determined that the quantitative score is at, or above, a predetermined threshold value.

In another aspect, a method is provided for confirming lymph node involvement in metastatic prostate cancer in a subject. The method includes administering to the patient an effective amount of a compound that selectively binds to prostate-specific membrane antigen (PSMA), the compound represented by Formula 1 or Formula 2 or a pharmaceutically acceptable salt thereof; determining a level of uptake of the compound in the prostate of the subject as a target (T) level; determining a level of uptake of the compound in control tissue as a baseline (B) level; and confirming lymph node involvement if a ratio of T:B is at, or above, a predetermined threshold value.

In another aspect, a method is provided for monitoring or assessing a status of prostate cancer in a human subject. The method may include determining a level of uptake of a gamma-emitting imaging agent comprising a prostate specific-membrane antigen (PSMA) recognition moiety and a radionuclide by at least a portion of prostate tissue of a human subject, which includes one or more tumor lesions; determining a ratio of (a) the level of uptake of said gamma-emitting imaging agent by said at least a portion of prostate tissue, and (b) a level of uptake of said gamma-emitting imaging agent by a control tissue of said human subject; and comparing said ratio to a baseline ratio previously determined for said human subject. In some embodiments, said ratio, if found to be higher than said baseline ratio, is indicative of disease progression. In some embodiments, said ratio, if found to be lower than said baseline ratio, is indicative of disease remission. In this and the method for confirming tumor metastasis with lymph node involvement in a prostate cancer patient, the compounds of Formula I and 2 are:

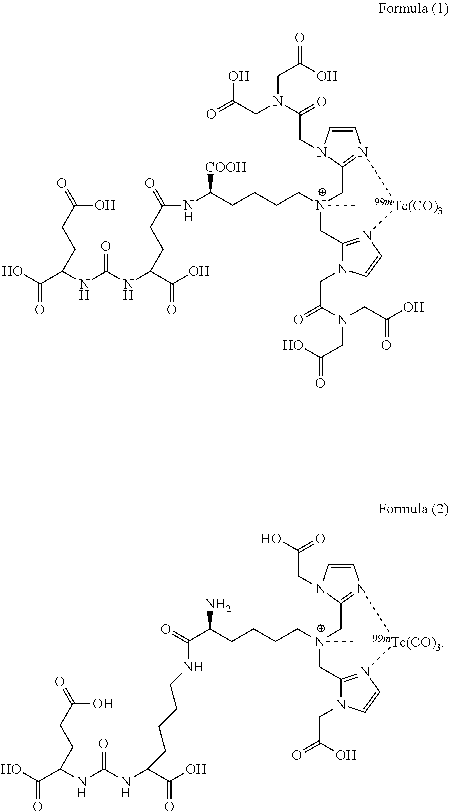

In the method, the predetermined threshold may be about 30. Formula (1) is alternatively known as trofolastat; 99mTc-trofolastat; MIP-^{99m}Tc-1404; 99mTc-MIP-1404; technetium Tc 99m trofolastat chloride; technetate(7-)-⁹⁹Tc, tricarbonyl[N²-[[[(1S)-1,3-dicarboxypropyl]amino]carbonyl]-L-γ-glutamyl-N⁶,N⁶-bis[[1-[2-[bis(carboxymethyl)amino]-2-oxoethyl]-1H-imidazol-2-yl-κN³]methyl)-L-lysinato(8-)-κN⁶]-, hydrogen, hydrochloride (1:7:1), (OC-6-33)-; or (OC-6-33)-tricarbonyl[N²-{[(1S)-1,3-dicarboxypropyl]amino]carbamoyl}-L-γ-glutamyl-N⁶,N⁶-bis[(1-{2-[bis(carboxymethyl)amino]-2-oxoethyl]-1H-imidazol-2-yl-κN³]methyl]-L-lysine-κN⁶](^{99m}Tc)(+)chloride.

In another aspect, a non-invasive method of assessing a degree of disease aggressiveness in a human subject diagnosed with prostate cancer is provided. The method includes recording a level of uptake of an effective amount of a gamma-emitting transition metal complex conjugated to a targeting moiety by diseased tissue of a human subject diagnosed with prostate cancer and determining from said level of uptake a degree of disease aggressiveness in said human subject. In some embodiments, said determination involves calculating a ratio of (a) the level of uptake of said gamma-emitting transition metal complex conjugated to a targeting moiety by said diseased tissue, and (b) a level of uptake of said gamma-emitting transition metal complex conjugated to a targeting moiety by a control tissue of said human subject. In some embodiments, the method also includes comparing the calculated ratio with a predetermined threshold. In some embodiments, the predetermined threshold is about 30. In some embodiments, the predetermined threshold is at least about 30. In other embodiments, the predetermined threshold is from 25 to 80. In yet other embodiments, the predetermined threshold is from about 25 to about 40. In any of the above embodiments, said gamma-emitting transition metal complex conjugated to a targeting moiety may be a compound that is MIP-^{99m}Tc-1404 or MIP-^{99m}Tc-1405. As used herein, aggressive disease is defined as disease having a Gleason score of >3+4, while statistically significant disease has a Gleason score of >3+3.

In another aspect, an in vivo method is provided for assessing a likelihood of a presence of metastatic disease in a human subject diagnosed with prostate cancer. The method may include recording a level of uptake of “^{99m}Tc-MIP-1404” by diseased tissue, which includes a primary tumor, of a human subject diagnosed with prostate cancer and determining from said level of uptake a likelihood of a presence of metastatic disease in said human subject. In some embodiments, said determination involves calculating a ratio of (a) the level of uptake of said gamma-emitting transition metal complex conjugated to a targeting moiety by said diseased tissue, and (b) a level of uptake of said gamma-emitting transition metal complex conjugated to a targeting moiety by a control tissue of said human subject. In some embodiments, the method also includes comparing the calculated ratio with a predetermined threshold. In some embodiments, the predetermined threshold is about 30. In some embodiments, the predetermined threshold is at least about 30. In other embodiments, the predetermined threshold is from 25 to 80. In yet other embodiments, the predetermined threshold is from about 25 to about 40. In any of the above embodiments, said gamma-emitting transition metal complex conjugated to a targeting moiety may be a compound that is ^{99m}Tc-MIP-1404 or ^{99m}Tc-MIP-1405.

In another aspect, a non-surgical method of diagnosing metastatic disease in a patient clinically diagnosed as having prostate cancer, which method does not rely on histopathology of a prostate or a lymph node is provided. The method includes administering to the patient an effective amount of a compound that selectively binds to prostate-specific membrane antigen (PSMA), the compound represented by Formula 1 or Formula 2 or a pharmaceutically acceptable salt thereof; determining a level of uptake of the compound in the prostate of the patient as a tumor (T) level; determining a level of uptake of the compound in a control tissue as a baseline (B) level; and confirming lymph node involvement if a ratio of T:B is at, or above, a predetermined threshold value. In the method, Formula I and Formula 2 are:

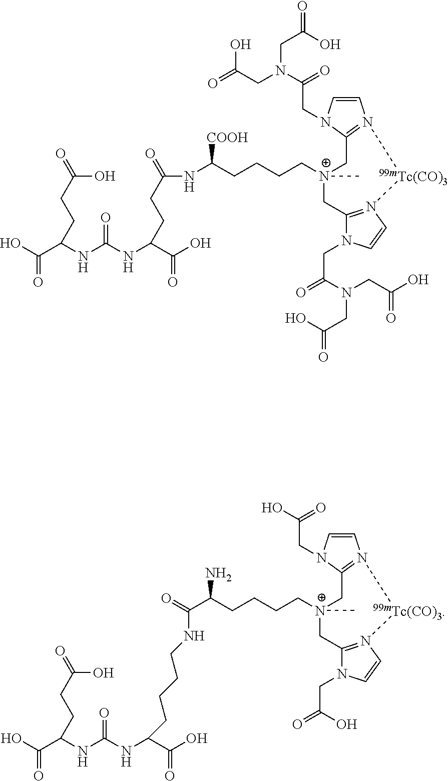

In some embodiments, the clinical diagnosis of prostate cancer is determined using a PSA value, digital rectal examination, trans-rectal ultra sound, symptomology, or a combination of any two or more thereof. In other embodiments, the predetermined threshold is about 30. In yet other embodiments, the T:B ratio is ≧30 and indicates a diagnosis of metastatic disease. In yet other embodiments, the T:B ratio is ≦30 and indicates a diagnosis of negative metastatic disease. The method may have a sensitivity of about 90%.

In any of the above methods, the human subject or patient may not have received prostate cancer treatment prior to conducting the method. Further, in any of the above methods, unless specifically identified, the determining includes obtaining an image of the patient using any of a number of nuclear medicine tomographic imaging techniques. Further, in any of the above methods, the T:B radio may correlate with a Gleason score. Further, in any of the above methods, the threshold value may be a surrogate marker for aggressive prostate disease. Further, in any of the above methods, the threshold value may be a surrogate marker for prostate metastasis.

In another aspect, a kit is provided that includes a first container including a free ligand MIP-1404, a second container including a ^{99m}Tc radionuclide, and instructions for producing ^{99m}Tc-trofolastat for: identifying a severity level of prostate cancer in a patient, confirming lymph node involvement in metastatic prostate cancer, confirming tumor metathesis, monitoring a status of prostate cancer, obtaining a SPECT/CT image of tissue expressing prostate-specific membrane antigen (PSMA) in vivo, detecting tumor metastasis to at least a portion of a bone or a soft tissue of a prostate cancer patient, identifying prostate tumor metastasis to a lymph node, monitoring the efficacy of prostate cancer treatment, monitoring or assessing a status of prostate cancer in a human subject, a non-invasive method of assessing a degree of disease aggressiveness in a human subject diagnosed with prostate cancer, assessing a likelihood of a presence of metastatic disease in a human subject diagnosed with prostate cancer, diagnosing metastatic disease in a patient clinically diagnosed as having prostate cancer, or identifying a severity level of prostate cancer in a patient harboring biopsy-confirmed prostate cancer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a graph of the clearance of compound represented by Formula 1 and Formula 2 from (A) blood and (B) urine and representative SPECT/CT scans (BL=bladder, LN=metathesis in lymph node), according to the examples.

FIGS. 2A and 2B show the biodistribution of compound represented by Formula 1 and Formula 2 in (FIG. 2A) a normal human subject and (FIG. 2B) a human subject with prostate cancer, according to the examples, compared to a standard bone scan (^{99m}Tc-MDP (methyldiphosphonate)).

FIG. 2C is a comparison of a Formula 1 scan with bone scans in a patient with metastatic prostate cancer. PSMA imaging with Formula 1 (in March) detected more metastatic lesions earlier compared to the two bone scans performed either before (in January) or after (in June) the PSMA scan, according to the examples.

FIGS. 3A-3D illustrates direct correlation between uptake of the compound represented by Formula (1) [^{99m}Tc-MIP-1404], in prostate cancer tissue imaged using SPECT and the Gleason score of tumor assigned by pathological analysis, according to the examples.

FIG. 4 is a histogram that correlates the Gleason score to measured expression of PSMA in prostate cancer lesions in subjects with prostate cancer, according to the examples.

FIG. 5 is a receiver-operator characteristic (ROC) determining the cutoff value for the target to background (T/B) ratio, according to the examples.

FIG. 6 Nomogram for predicting positive Lymph Node Involvement (LNI), according to the examples.

FIG. 7 compares examples of histologically confirmed primary prostate lesions as seen in fused axial ^{99m}Tc-MIP-1404 SPECT/CT reconstructions from four study patients (row A), and matching axial T1W MRIs (row B), arranged by Gleason score from left to right, according to the examples.

FIG. 8 Illustrates the quantitative T:B ratio from a prostate gland determined from the maximum count value within the gland: background mean count value for the obturator muscle as analyzed by a SPECT/CT image from a circular region of interest (ROI; in this figure the pelvic region with the prostate shown) within a 2 cm diameter, according to the examples.

FIG. 9A is a histogram of reader scores determined using the Prostate Scoring Scale, a semi-quantitative measurement (Table 9) of the uptake of the compound represented by Formula (I) (^{99m}Tc-MIP-1404) in prostate lobe tissue imaged using SPECT correlated with Gleason Score (ρ<0.0001) and Spearman's rank order correlation coefficient (ρ=0.476), according to the examples.

FIG. 9B is a histogram of quantitative scores for T:B ratios based upon the maximum count value within the prostate: mean count value for the background, both from a circular ROI of 2 cm diameter of the uptake of the compound represented by Formula (I) (^{99m}Tc-MIP-1404) in prostate lobe tissue imaged using SPECT correlated with Gleason Score (ρ<0.0001) and Spearman's (ρ=0.504), according to the examples.

FIG. 10A is a graph of ROC Analysis (scores per prostate lobe) for semi-quantitative (reader) measurements, and showing that reader discriminate lobes with ≧3+3 and ≧3+4 from normal lobes better than quantitation alone, according to the examples.

FIG. 10B is a graph of ROC Analysis (scores per prostate lobe) for quantitative T:B ratios, and showing better discrimination with quantitation in high grade disease from normal lobes than reader semi-quantitative scores, according to the examples.

FIG. 10C is a graph of ROC analysis illustrating that a T:B cutoff of about 30 in the primary prostate tumor may be used to diagnose lymph node metastasis of primary prostate cancer, according to the examples.

FIG. 11A is a graph showing the mean PSA values in prostate cancer patients who received therapy prior to administration of the compound represented by Formula (I) (^{99m}Tc-MIP-1404), according to the examples.

FIG. 11B is a histogram of the mean quantitative T:B ratios of the update by the prostate gland of ^{99m}Tc-MIP-1404 in patients (Tx) who received prostate cancer therapy prior to injection and imaging using tissue imaged using ^{99m}Tc-MIP-1404, compared to patients (no Tx) who had not received prostate cancer therapy prior to injection and imaging, according to the examples.

FIG. 12A compares fused axial ^{99m}Tc-trofolastat SPECT/CT reconstructions (left), and axial T1W MRI (right). Arrows indicate a histologically confirmed positive 6 mm right obturator lymph node read as positive by ^{99m}Tc-trofolastat SPECT/CT readers and positive by the MR reader, according to the examples.

FIG. 12B compares fused axial ^{99m}Tc-MIP-1404 SPECT/CT reconstruction (A), and axial T1W MRI (B), indicating a histologically confirmed positive lymph node read (5 mm left hypogastric lymph node) as positive by the SPECT/CT reader and negative by the MR reader, according to the examples.

FIG. 13 illustrates the detection of skeletal disease involvement through the comparison of a whole-body planar bone scan and ^{99m}Tc-MIP-1404 scan, according to the examples.

FIG. 14 illustrates the prostate scoring regions as used with the Lesion Visualization Grading Score to analyze ^{99m}Tc-MIP-1404 SPECT/CT images, according to the examples.

FIG. 15 illustrates the pelvic lymph node scoring regions as used with the Lesion Visualization Grading Score to analyze ^{99m}Tc-MIP-1404 SPECT/CT images, according to the examples.

FIG. 16 is a graph of the statistical correlation of tumor:background ratio calculated from ⁹⁹mTc-MIP-1404 uptake compared with Gleason Score in lobes of the prostate (ρ<0.0001), according to the examples.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The imaging of prostate cancer (PCa) to differentiate cancerous tissue from indolent disease within the prostate gland is challenging. Also challenging is the identification of metastatic and recurrent tumors using routine clinical imaging methodologies. Current methods for detection and imaging of prostate cancer rely on a combination of PSA score, needle biopsies, MRI, bone scan, and Gleason scores. The present technology uses compounds that bind with high selectivity to PSMA, a zinc metalloprotein that is overexpressed on all prostate cancer cells, higher grade prostate tumors, metastatic disease, hormone refractory prostate cancer, as well as the neo-vasculature of other solid tumors. PSMA targeting compounds disclosed herein demonstrate high sensitivity, specificity, and accuracy, have significant advantages over current methods, and offer the potential to replace them as the primary diagnostic/prognostic agent of choice. Further, it is shown that through statistically significant analysis of the uptake of PSMA targeting compounds by the prostate a strong, statistically significant correlation to Gleason score may be obtained. The correlation may also be used as a non-invasive (i.e. no surgery or prostate biopsy) measure of determining or diagnosing if cancer is present, the extent of the cancer in the gland, if the cancer has undergone metastasis with lymph node involvement.

^{99m}Tc-labeled anti-PSMA inhibitors, Formula (1) and Formula (2) compounds (^{99m}Tc-MIP-1404 and ^{99m}Tc-MIP-1405 respectively), structurally illustrated below are highly specific radiolabeled agents for imaging PCa. The compound represented by Formula (1) is a glutamate-urea-glutamate based dimer while the compound represented by Formula (2) is a glutamate-urea-lysine heterodimer.

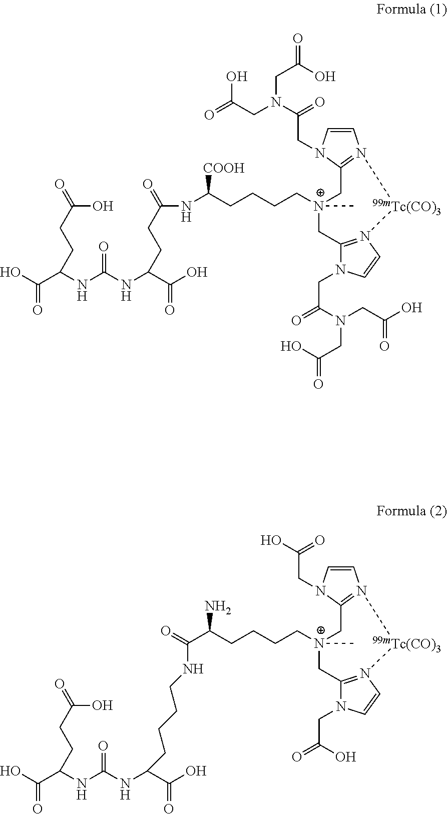

As illustrated, the dimeric backbone of both Formula (1) and Formula (2) compounds contain carboxylate residues that bind to the basic substrate binding pockets of the protein. The radiolabel chelator is attached to the side chain carboxyl residue (Formula (1)) or the side chain amine group (Formula (2)) through an intervening linker. In vitro binding studies show the compound represented by Formula (I) to bind PSMA with an affinity of 104 nM while the compound represented by Formula (2) binds to PSMA with an affinity of 31 nM.

In vivo pharmacokinetic and biodistribution studies show that the compounds represented by Formula (1) and Formula (2) accumulate in the liver, kidneys and salivary glands as well as the urinary bladder and prostate tissue. Uptake in liver and kidney tissue is greater for the compound represented by Formula (1) than the compound represented by Formula (2), however, physiological clearance rate is more rapid for the compound represented by Formula (2) than for the Formula (1) compound.

As illustrated herein, ^{99m}Tc-trofolastat (see below), is one radioactive diagnostic agent that may be useful in diagnosing patients with biopsy-confirmed prostate cancer as an aid to identifying the severity of the disease in the patent. Also, as illustrated herein, ^{99m}Tc-trofolastat (see below), is a radioactive diagnostic agent that may be useful in diagnosing patients with prostate cancer, and as an aid to identifying not only the severity of the disease in the patent, but the likelihood of metastasis of the disease. The compound may also be used to help determine patient treatment options.

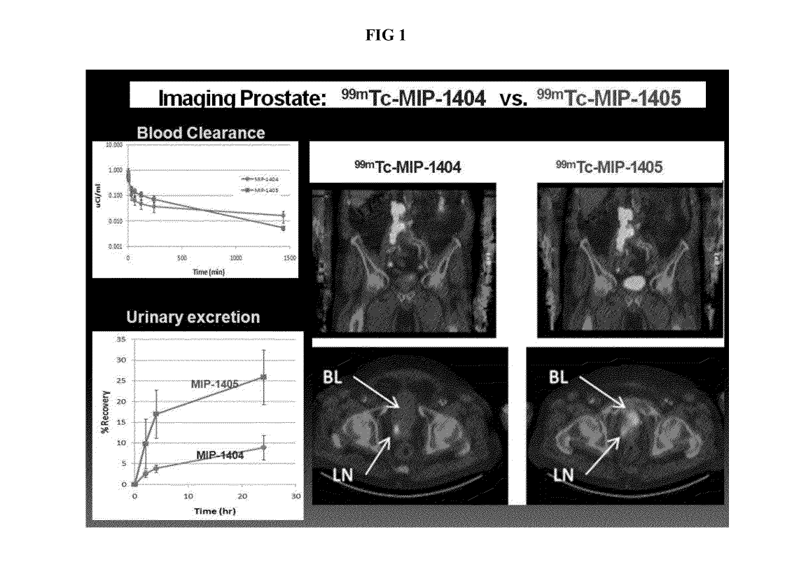

FIG. 1A is an illustration of the blood clearance rates for compound represented by Formula (1) and Formula (2). While both compounds are cleared from blood over a period of about 1500 minutes the rate of clearance of the Formula (2) compound is greater than the rate of clearance of the compound represented by Formula (1)S. The present inventors also measured the amount of Formula (1) and Formula (2) compounds excreted in urine samples of patients over a time period of 30 hours post administration. As illustrated in FIG. 1B a significantly greater amount of the Formula (2) compound was present in urine. Taken together, these observations suggest that compound represented by Formula (2) is more rapidly cleared from the body than the compound represented by Formula (1). While rapid clearance of a radioimaging agent is desirable, the time period a radioimaging agent resides in the body is also important for proper imaging.

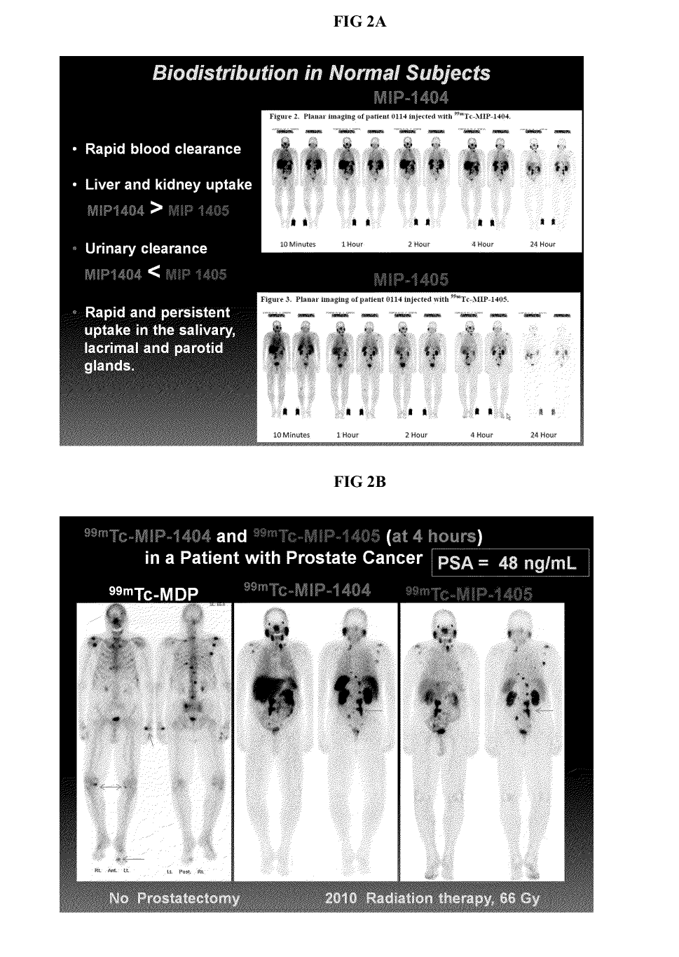

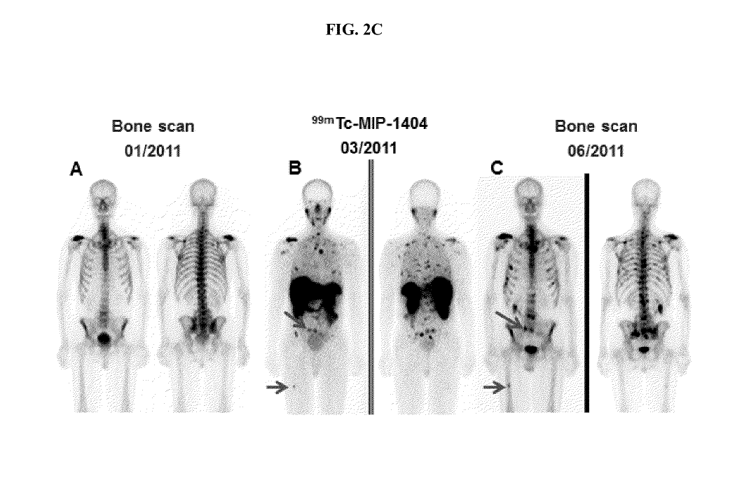

FIG. 2A and FIG. 2B illustrate full body scans of normal and cancer patients at various intervals of time over a 24 hour period, post administration of a Formula (1) or a Formula (2) compound. While both compounds rapidly concentrate in the liver, kidney, urinary bladder, prostate, lacrimal glands, lymph nodes and salivary glands within 10 minutes of administration, the compound represented by Formula (2) clears more rapidly from these organs than the compound represented by Formula (1). For instance, full body scintigraphy (scans) of patients receiving the compound represented by Formula (1) at 4 hours post administration showed a weaker intensity of gamma radiation signal in the liver, kidney, urinary bladder, prostate, lacrimal glands and salivary glands, with near complete loss of gamma radiation signal in scintigraphic images at the 24 hour time point.

Full-body scintigraphic images using a Formula (1) compound clearly illuminates the prostate, lymph nodes, liver and kidneys in the image at 4 hours post administration. SPECT/CT images of patients at 4 and 24 hours show excellent contrast for lesion versus background tissue. The percent intensity of signal detected as a function of drug administered is greater for the compound represented by Formula (1) than Formula (2) at every time point at which detection was carried out.

Detection of tumor metastasis to the bone or soft tissue is evident earlier during the clinical course of the cancer with the compound of Formula (1) as compared to other conventional radionuclide imaging agents used in the clinic. See FIG. 2C. Because imaging with the compound of Formula (1) permits early detection of tumor and metastasis, early therapeutic interventions may be possible to stem the progress and spread of prostate cancer.

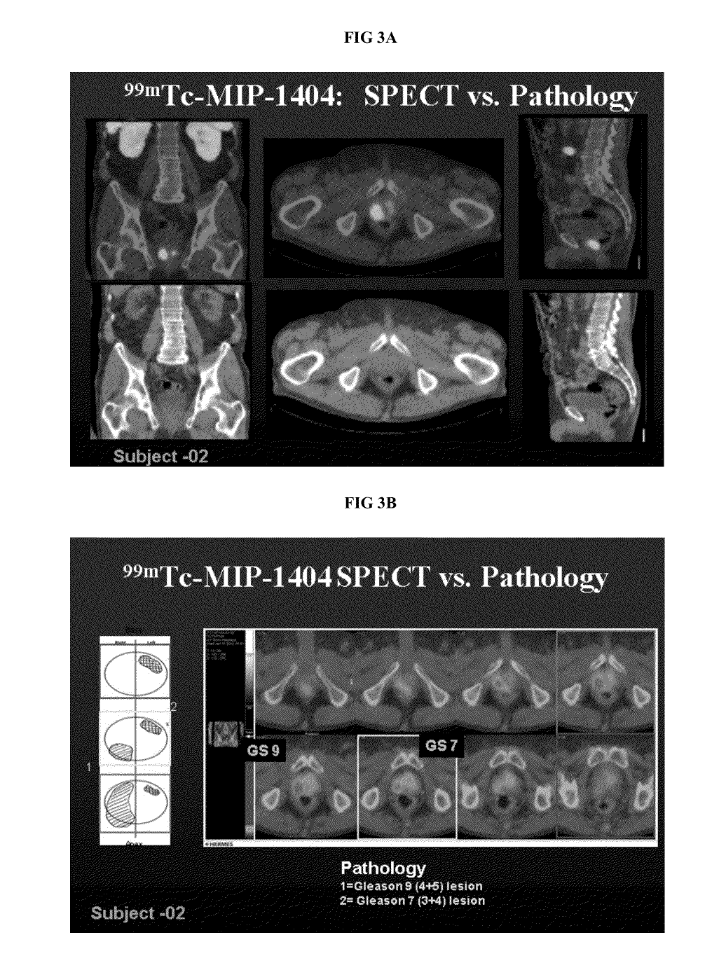

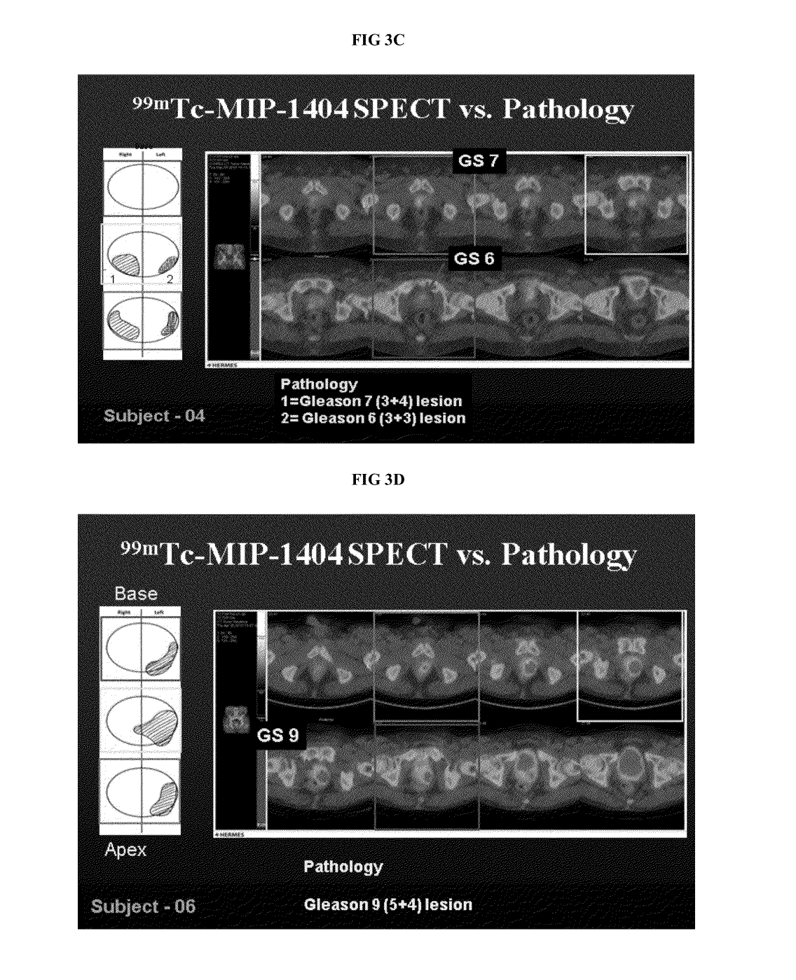

Imaging of lesions (tumor) using the ^{99m}Tc radioimaging agents of the present technology depends on the PSMA levels expressed on the surface of cancerous tissue. As mentioned above, compounds of Formula (1) and Formula (2) contain a targeting moiety that selectively binds to PSMA. Expression of PSMA also correlates to the grade of prostate cancer. The Gleason score that is used as a prognostic marker for the aggressiveness of prostate cancer is based on the grade of prostate cancer obtained by histopathological analysis. The present inventors have shown that the uptake levels of both compounds directly correspond with the Gleason score. The correlation between ^{99m}Tc uptake levels and the Gleason score was stronger for the compound according to Formula (1) than the compound according to Formula (2). That is, prostate tumors with a higher Gleason score show greater uptake when the compound according to Formula (1) is the radioimaging agent. See FIGS. 3A-3D.

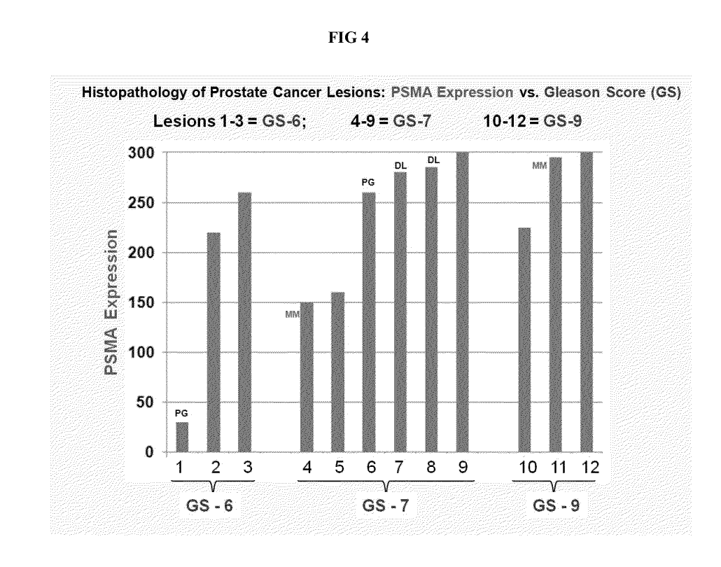

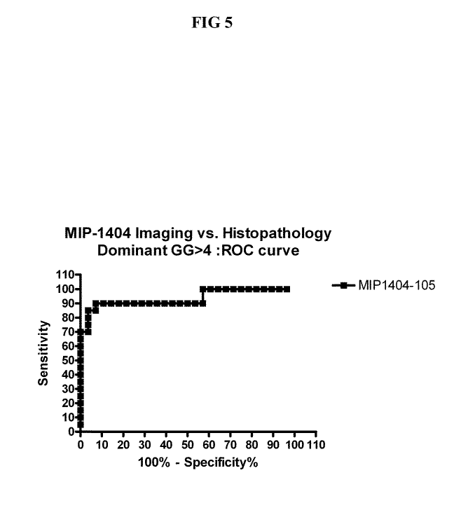

The correlation between a higher Gleason score and greater ^{99m}Tc uptake levels in prostate cancer tissue existed in all prostate cancer patients enrolled in a study by the inventors. FIG. 4 shows a histogram that correlates tissue PSMA expression levels to the Gleason score. As illustrated, three groups of cancer patients with a Gleason score of 6, 7 or 9 were studied. A greater Gleason score corresponds to a greater expression of PSMA. Because compounds represented by Formula (1) and Formula (2) contain a PSMA targeting moiety, the greater the expression of PSMA, the greater will be the uptake levels of these radioimaging agents.

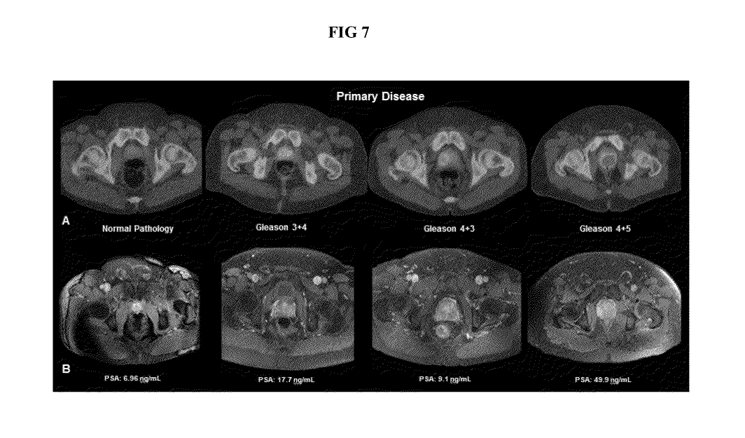

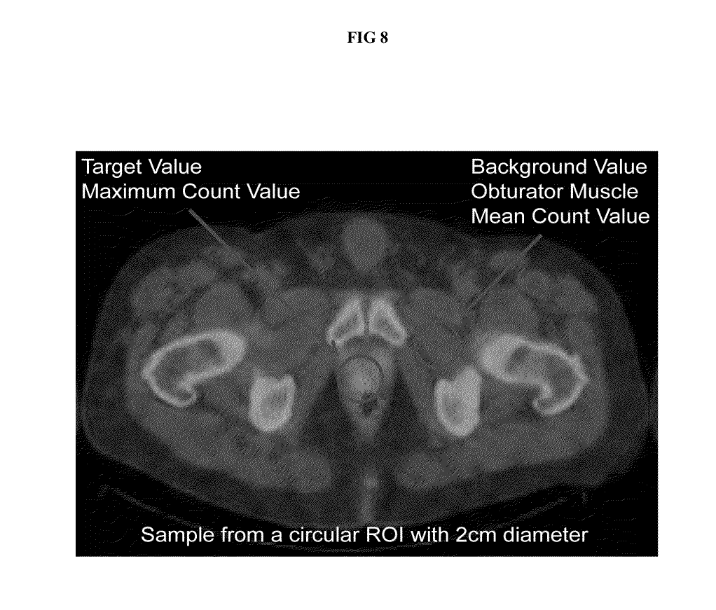

Table 1 provides a correlation of the Gleason score of eight prostate cancer patients to the ratio of ^{99m}Tc uptake in tumor tissue (T) to normal tissue (background (B)). As illustrated in FIG. 7, the lack of focal uptake of the compound represented by Formula (1) in normal prostate tissue or other normal tissue (A, normal pathology), further demonstrates PSMA as a viable target for detection and visualization of prostate cancer. The ratio of tumor uptake to background (T/B ratio), moreover, was observed to directly correlate with the Gleason score. This correlation provides a rationale for replacing conventional prostate biopsies for determination of Gleason scores, with the method provided herein for determination of prostate cancer and the extent of the disease.

Thus, a T/B ratio in the range from about 5.9 to about 13.0 corresponds to a Gleason score of 7. A T/B ratio of about 13.1 to about 15.4, for example a T/B ratio of 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, or 15.4 correspond to a Gleason score of 8. A T/B ratio in the range from about 15.5 to about 45, about 16 to about 44, about 17 to about 43, about 18 to about 42, about 19 to about 41, about 20 to about 40 correspond to a Gleason score of 9.0. Thus, T/B ratios of about 21, 22, 23, 24, 25, 26, 27, 28, 29, 30, 31, 32, 33, 34, 35, 36, 37, 38, 39, or 40 correspond to a Gleason score of 9.0.

TABLE 1
^{99m}Tc-MIP-1404 (Formula (1): Gleason Score V/S T/B Ratio
(Technetium Tc 99m Trofolastat chloride - USAN)

| | Gleason | ^{99m}Tc Tumor (T) | ^{99m}Tc | T/B | Clinical |
|---|---|---|---|---|---|
| Subject | Score | Max Counts | Background (B) | Ratio | Reading |
| 2 | 9 | 1550 | 100 | 15.5 | 3 |
| 6 | 9 | 3300 | 100 | 33 | 4 |
| 9 | 9 | 4500 | 100 | 45 | 4 |
| 3 | 7 | 780 | 60 | 13 | 0 |
| 4 | 7 | 700 | 50 | 14 | 1 |
| 8 | 7 | 1050 | 100 | 10.5 | 3 |
| 11 | 7 | 750 | 75 | 10 | 1 |
| 12 | 7 | 650 | 100 | 6.5 | 1 |

The T/B ratio is useful for staging prostate cancer. Briefly, prostate cancer patients undergo full body imaging post administration of a compound represented by Formula (1). The images are used to quantitate the level of uptake of the Formula (1) compound in cancer tissue and normal tissue. The amount of Formula (1) compound in prostate tissue is divided by the amount of Formula (1) in normal tissue to arrive at a T/B ratio. In one embodiment a standard curve that correlates a numerical value of T/B to the stage of a prostate cancer on a scale of I-IV is used for staging the cancer in the test subject. Based on the T/B ratio, prostate cancer patients with a stage cT3 or CT4 cancer are enrolled in a clinical study aimed at developing a nomogram that will be used to discriminate and calibrate the probability of a prostate cancer patient having Lymph Node Invasion (LNI). The development of such a nomogram is further illustrated below.

The T/B ratio also is useful for monitoring the status of prostate cancer in a human subject. Briefly, the human subject is administered an effective amount of a compound of Formula (1) or Formula (2). The subject undergoes imaging at 1-4 hours post administration of the compound. One or more images of the pelvic region or full body scans may be obtained during imaging. Moreover, the subject may be imaged at regular intervals of time post administration of the imaging agent. Illustratively, the subject may be imaged at 1, 2, 3, 4, 5, 6, 7 8, 9, 10, 12, 14, 16, 18, 20, 22, or 24 hours.

The level of uptake of the compound by at least a portion of prostate tissue is measured and compared to a level of uptake by control tissue, so as to determine a ratio of the level of uptake of the compound by at least a portion of prostate tissue to the level of uptake by control tissue. This ratio is then compared to a baseline ratio previously determined for the human subject. The normal tissue may be any tissue, for example, non-tumorous portions of prostate tissue, normal pelvic lymph node tissue, or pelvic muscle tissue.

The status of a subject harboring prostate cancer according to the method of the invention is deemed to have worsened if the ratio is above the baseline ratio. Typically, an elevated risk of systemic dissemination and death are associated once the cancer metastasizes to the pelvic lymph nodes. Clinically this phenomenon is called Pelvic Lymph Node Involvement (LNI). Nomograms are used to estimate the likelihood of occult nodal disease and guide clinical decisions with regards to therapeutic options. The T/B ratios may also be used for determining lymph node involvement in metastasis, where the ratio is at least about 30.

According to an aspect of the method, a nomogram was developed to predict the status of a subject with prostate cancer using pre-treatment PSMA levels, T/B ratio, biopsy Gleason score, stage and LNI as variables. The development of the nomogram is further explained below. Briefly, points are assigned for specific values associated for each variable of the nomogram and a total point score is calculated for the patient. The total point score is then used to calculate the probability of LNI. A greater probability of LNI indicates a worsening status for the subject with prostate cancer.

As noted above, the compound represented by Formula (1) or Formula (2) are suitable for use as radio-imaging agents for imaging PSMA expressing prostate cancer cells. Accordingly, in one embodiment, a pharmaceutical composition is provided that includes a compound represented by Formula 1 or Formula 2 or a salt, stereoisomer, or tautomer thereof, and a pharmaceutically acceptable carrier.

In general, the compound represented by Formula 1 or Formula 2 or pharmaceutical compositions thereof, are administered parenterally, usually by injection. Parenteral routes include, but are not limited to, intravenous, intramuscular, intraarterial, intrathecal, intracapsular, intratumoral, intradermal, intraperitoneal, subcutaneous, intraarticular, and infusion.

The pharmaceutical composition provided is suitable for in vivo imaging. Accordingly, in another embodiment the use of radiotherapeutic agents is provided for the treatment of prostate cancer patients whose progression of disease and extent of metastasis is diagnosed using the compound represented by Formula 1 or Formula 2. Thus, suitable pharmaceutical compositions may contain a radio imaging agent, or a radiotherapeutic agent that has a radionuclide either as an element, i.e. radioactive iodine, or a radioactive metal chelate complex in an amount sufficient for therapy, together with a pharmaceutically acceptable radiological vehicle. The radiological vehicle should be suitable for injection, such as aqueous buffer solutions, e.g., tris(hydromethyl)aminomethane (and its salts), phosphate, citrate, bicarbonate, etc.; sterile water; physiological saline; and balanced ionic solutions containing chloride and or dicarbonate salts or normal blood plasma cations such as calcium, potassium, sodium, and magnesium.

The concentration of the imaging agent in the radiological vehicle should be sufficient to provide satisfactory imaging. For example, when using an aqueous solution, the dosage is about 1.0 to 50 milliCuries. The actual dose administered to a patient for imaging or therapeutic purposes, however, is determined by the physician administering the imaging agent. The imaging agent should be administered so as to remain in the patient for about 1 to 24 hours, although both longer and shorter time periods are acceptable. Therefore, convenient ampoules containing 1 to 10 mL of aqueous solution may be prepared.

Imaging may be carried out in the normal manner, for example by injecting a sufficient amount of the imaging composition to provide adequate imaging and then scanning with a suitable machine, such as a gamma camera. In certain embodiments, a method of imaging a region in a patient, for example, imaging one or more tissues that express prostate-specific membrane antigen (PSMA) includes the steps of: (i) administering to a patient a diagnostically effective amount of a compound represented by Formula 1 or Formula 2 so as to contact the one or more tissues expressing PSMA; and (ii) recording a radiographic images of the one or more tissues. In one embodiment the tissue imaged is a prostate tissue or a prostate cancer tissue. In another embodiment, the tissues imaged are pelvic lymph node tissues. In yet another embodiment, the tissue imaged is bone tissue.

Overexpression of PSMA, a measure of the aggressiveness of a prostate cancer, is directly correlated to the Gleason score. A direct correlation also exists between the T/B ratio and the Gleason score. Based on the T/B ratio a physician may select the most appropriate therapeutic regimen for treatment. In one aspect, small molecule compounds that selectively bind PSMA and carry an appropriate radionuclide, for example, ¹³¹Iodine, ¹⁹²Iridium, ¹⁸⁶Rhenium, or ²¹²Lead can be used to selectively treat prostate cancer.

The radiopharmaceutical can be administered as a stable pharmaceutical composition parenterally, usually by injection. In one aspect, the present invention provides combination therapy in which a patient or subject in need of therapy is administered a radiopharmaceutical in combination with chemotherapy, anti-androgen therapy or both.

A therapeutically effective dose of the radiopharmaceutical may be administered separately to a patient or subject in need thereof from a therapeutically effective dose of the combination drug. The person of skill in the art will recognize that the two doses may be administered within hours or days of each other or the two doses may be administered together.

In one embodiment, pharmaceutical compositions are provided that are suitable for single unit dosages that include a radiopharmaceutical, its pharmaceutically acceptable stereoisomer, prodrug, salt, hydrate, or tautomer and a pharmaceutically acceptable carrier.

Compositions suitable for parenteral administrations are administered in a sterile medium. Depending on the vehicle used and the concentration of the drug in the formulation, the parenteral formulation can either be a suspension or a solution containing dissolved drug. Adjuvants such as local anesthetics, preservatives and buffering agents can also be added to parenteral compositions.

The present invention, thus generally described, will be understood more readily by reference to the following examples, which are provided by way of illustration and are not intended to be limiting of the present invention.

### EXAMPLES

### General Protocol for Assessing the Diagnostic Accuracy by Imaging with a Formula (1) or a Formula (2) Compound

A phase-2 study to image men with high-risk prostate cancer scheduled for radical prostatectomy (RP) and Extended Pelvic Lymph Node Dissection (EPLND) was performed using a compound according to Formula (1), as an illustrative radioimaging agent. The primary objective of the study was to assess the safety and the ability of the Formula (1) compound to detect prostate cancer within the prostate gland. Secondary objectives include (i) assess the ability of the Formula (1) compound to detect the extent and location of prostate cancer within the prostate gland, (2) assess the ability of the Formula (1) compound to detect metastatic PCa within pelvic lymph nodes and to further detect the specific location of metastatic PCa within anatomic pelvic lymph node regions, (3) compare the performance of the Formula (1) compound as a prostate cancer imaging agent to MRI and compare the ability of the Formula (1) compound to detect the specific location of metastatic PCa within pelvic lymph nodes to the ability of MRI for detecting the specific location of metastatic PCa within pelvic lymph nodes.

### Study Design.

A Phase-2 multi-center, multi-reader, open-label trial, to assess the performance characteristics of the Formula (1) compound as an imaging agent was measured by true-positive fraction (TPF equivalent to sensitivity) and false-positive fraction (FPF, equivalent to 1-specificity). The “truth standard” for determining true-positive cases and false-positive cases were histopathology results obtained subsequent to RP and EPLND. The performance of the Formula (1) compound as an imaging agent was compared to MRI by calculating (1) the difference in correctly identified positive cases by each imaging method that also are positive by histopathology subsequent to RP and EPLND (TPF); and (2), the difference in incorrectly identified negative cases by each imaging method, that also are negative by histopathology subsequent to RP and EPLND (FPF).

Newly-diagnosed prostate cancer patients at high-risk for metastatic disease who were scheduled for RP with EPLND were enrolled in the study. Subjects had an MRI as part of study's screening protocol. Subjects will receive a single intravenous dose of the Formula (1) compound (study drug) followed by both whole-body planar and SPECT/CT imaging 3-6 hours after injection. As standard of care, subjects underwent RP with EPLND surgery and histological assessment of specimens no more than 3 weeks after study drug dosing. Images of the patients were evaluated for visible uptake of the Formula (1) compound within the prostate gland and by regional assessment of nodal disease. These findings were compared against histopathology results used as the truth standard.

Clinical, imaging and pathology staff members responsible for handling surgical specimens remain were blinded to all images obtained using the Formula (1) compound prior to completing surgery and/or reporting of histopathology results. Based on an estimate that 20% of high-risk subjects will have metastatic prostate cancer in regional lymph nodes, approximately 100 evaluable subjects were enrolled in the trial.

Subjects enrolled in the study met all of the following criteria:
1. Male aged 21 years or older,
2. Ability to provide signed informed consent and willingness to comply with protocol requirements,
3. Biopsy confirmed presence of adenocarcinoma of the prostate gland,
4. At high-risk for metastatic disease by a stage of cT3, cT4, or a total nomogram score of greater than or equal to 130,
5. Scheduled to undergo radical prostatectomy with extended pelvic lymph node dissection,
6. Agree to use an acceptable form of birth control for a period of 7 days after the injection of a Formula (1) compound.

Subjects meeting the following criteria, moreover, were excluded from participating in the study:
1. Participating would significantly delay the scheduled standard of care therapy,
2. Administered a radioisotope within 5 physical half-lives prior to injection with the study drug,
3. Have any medical condition or other circumstances that, in the opinion of the investigator, would significantly decrease obtaining reliable data, achieving study objectives or completing the study,
4. Have a contraindication for MR imaging.

The injection of the Formula (1) compound was administered as an intravenous bolus. A normal saline flush (˜10 mL) was used to ensure complete administration of the Formula (1) compound. The duration of subject participation will be from the time of signing informed consent through the pre-surgery procedures on the day of prostatectomy surgery. Subjects will be deemed enrolled in the study once the subject signs informed consent and receives an injection of the compound represented by Formula (1). Standard of care RP will be performed no more than 3 weeks following the administration of an injection of the compound represented by Formula (1). All tissue collections occurred as part of the subject's standard of care.

The safety of study participants was evaluated by reviewing occurrences of adverse events, changes in the vital signs of the participant and changes to values of the clinical measurements upon administration of the compound represented by Formula (1).

Efficacy analyses were conducted utilizing histopathology results subsequent to radical prostatectomy and extended pelvic lymph node dissection as the truth-standard for determination of positive and negative cases. Primary efficacy analyses estimated the ability of the compound represented by Formula (1) to detect cancer in prostate glands that were confirmed as harboring tumor based on a biopsy. The primary efficacy analysis evaluated sensitivity and specificity of the compound represented by Formula (1) using 80% power to establish the lower bound of one-sided 95% confidence intervals. All subjects who receive the Formula (1) and complete surgery will be included in the primary efficacy analysis.

### Example 1

Pharmacokinetics, Biodistribution, Dosimetry, Metabolism & Excretion of compound represented by Formula (1) and Formula (2). Methods: Fourteen subjects (7 metastatic prostate cancer patients and 7 healthy, normal males) were enrolled in a phase 1, single-blind, randomized, cross-over study in which subjects were randomly administered a single dose (740 MBq; 20 mCi) of a compound represented by Formula (1) and a similar chemical analog, a compound represented by Formula (2), 14 days apart. Both Formula (1) and Formula (2) compounds displayed pharmacokinetic and distribution characteristics including tumor uptake and retention with clearance rates that are suitable for radioimaging agents. Dosimetry studies confirmed that the estimated radiation dose for both compounds is within the clinically acceptable range for a diagnostic radiopharmaceutical. (FIGS. 1 and 2A)

Results:

The ^{99m}Tc-containing Formula (1) in particular displayed favorable clearance and tumor to background ratio with minimal accumulation in the urinary ladder bladder. The compound represented by Formula (1) rapidly localized to lesions in lymph nodes and bone as visualized by whole-body imaging as early as 1 hour post-injection in men with prostate cancer. Single-photon emission computerized tomography (SPECT/CT) images at 4 and 24 hours demonstrated excellent lesion contrast with target to background ratios ranging from 3:1 to 28:1 at 4 and 24 hours respectively. Enlarged and sub-centimeter lymph nodes were also clearly visualized. (FIG. 2B)

In a 71 year old patient who had prior prostatectomy and with a rising PSA (1.37-8.9 ng/ml over a period of 4 months), both Formula (1) and (2) agents identified multiple foci of metastatic cancer not seen in the bone scan obtained only 2 months earlier (FIG. 2C). A repeat bone scan obtained 3 months after the ^{99m}Tc study, however showed multiple foci of bone lesions. This observation suggests that PSMA targeted molecular imaging may identify disease progression earlier than the standard bone scan. In addition, in several patients, significant uptake was also observed in lymph nodes smaller than 10 mm, considered normal by size threshold criteria used in cross-sectional imaging such as CT and MR. Such observations suggest an improvement in the sensitivity of lesion detection with molecular imaging using small molecule ^{99m}Tc labeled PSMA inhibitors.

### Example 2

Protocol for Determining the Optimal Threshold Value for Discriminating Low Grade Prostate Cancer from a Higher Grade Prostate Cancer. Methods: SPECT/CT images of the pelvis including the prostate gland were obtained with a hybrid gamma camera in a 128×128 pixel matrix format with a 360 degree circular or elliptical orbit acquired into 120-128 frames. Raw images were reconstructed into 3D space with an iterative ordered subset estimation maximization algorithm corrected for attenuation and resolution recovery. Axial slices of the 3D volume were displayed with a HERMES H-SMART™ workstation (HERMES Medical Solutions; Stockholm, Sweden). Circular regions of interest with a diameter of approximately 20 pixels were placed on the obturator muscle adjacent to and to the left side (patient left) of the prostate gland. Counts within that region were recorded as background. Axial slices through the lower third, middle and upper third of the gland were selected to sample the apex, mid-gland and base of the prostate respectively. Radioactivity counts form the right and left side of the gland for each of the three slices were obtained for the same sized circular region of interest as background. The target to background ratio (T/B) was obtained by dividing the counts from prostate tissue by the background count. When large intense lesions originating in one side of the gland crossed the midline due to morphological changes to the anatomy, the area was scored according to the site of origin.

Results:

Target to background ratios for all patients were compared against a truth standard which consisted of step-section histopathology analysis to obtain the total Gleason score and primary Gleason grade in approximately the same location of the prostate gland. A receiver operator characteristic (ROC) curve was generated (Graph Pad Software; La Jolla, Calif.) with the Gleason score and primary Gleason grade values of ≧7 and ≧4 respectively. See FIG. 5. It was determined that the optimal cutoff value for target to background ratio within a region of the prostate gland demonstrating the highest accuracy and balance of sensitivity and specificity for discriminating low grade disease from a higher grade disease was 5.9. This value was also consistent with observations in normal healthy volunteers obtained in earlier clinical trials which typically had a segmental target to background value of <6 (data not shown).

### Example 3

### Methods

Patients (n=8) diagnosed with localized PCa (Gleason ≧7 with ≧3 biopsy cores positive, and at least one core ≧30% involved with PCa) and who were scheduled for radical prostatectomy (RP) participated in this Phase 1 study. Within two weeks of surgery, each subject received a single dose of a compound represented by Formula (1), (20 mCi), and was then subjected to a planar whole body and Single Photon Emission Computed Tomography (SPECT) images between 2-4 hours post injection (p.i.). The uptake of the Tc-99m in prostate lesions was quantified, and the imaging results were compared to CT/MRI, histopathology and PSMA staining.

Results:

All subjects completed the study yielding 60 evaluable prostate sectors and greater than 80% of the sectors contained a PSMA+PCa nodule. The dominant tumor nodule was detectable by SPECT imaging in all patients and correlated with pathological location within the prostate. The lesion detection, in part, depended upon both PSMA expression and tumor volume. The tumor/background ratio was 10.8±2.2 with a Gleason score of 7, and this ratio was 30±10 with a Gleason score of 9. In all subjects with a Gleason score ≧7, ^{99m}Tc-MIP-1404 SPECT clearly identified the PCa foci confirmed by histopathology and PSMA staining.

The small molecule PSMA inhibitor represented by Formula (1) rapidly detects primary and metastatic PCa with high specificity. The ^{99m}Tc uptake in the lesions correlated well with both Gleason score and PSMA expression. The PSMA based small molecule SPECT imaging probe visually distinguishes aggressive from indolent disease as evidenced by the trend towards improved detection with increasing Gleason grade.

### Example 4

### Methods

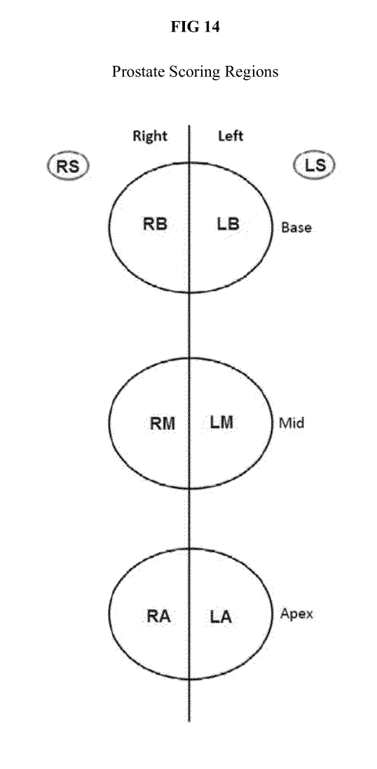

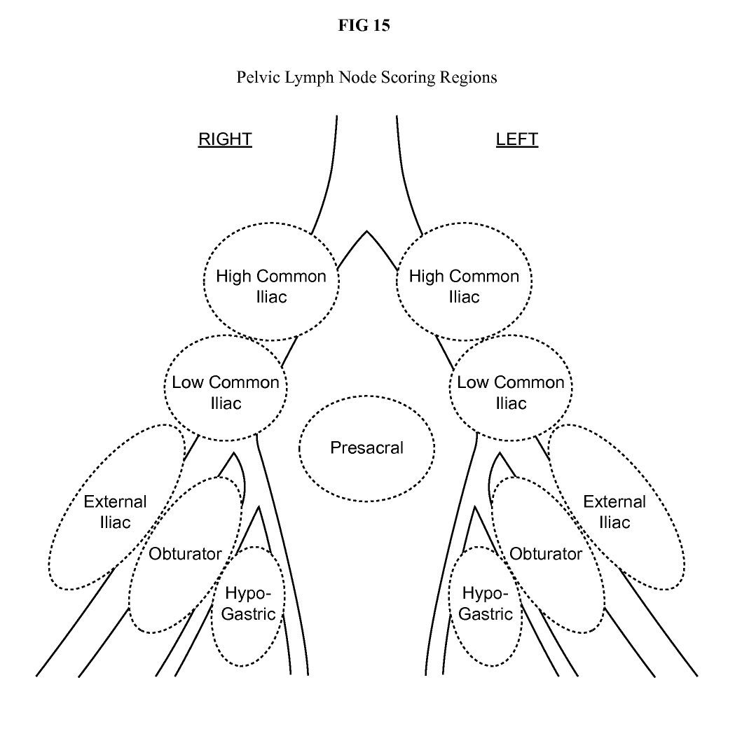

Patients (pts) with biopsy confirmed adenocarcinoma of the prostate scheduled for RP with extended pelvic lymph node dissection (EPLND) at high risk for disease outside of the prostate gland were eligible. High risk patients were stage of T3c or T4c or a nomogram score ≧130 (Godot′ et al., Eur. Urol., (2011), p 195-201). Within 30 days of screening, the patients required a bone scan and pelvic MRI. After enrollment, the patients received a compound represented by Formula (1) at a dose of 20 mCi±3 mCi followed by whole-body planar and SPECT/CT imaging 3 to 6 hrs later. Patients then underwent RP with EPLND within 21 days. SPECT/CT images were evaluated centrally by 3 readers blinded to clinical information and compared to on-site pathology assessments using a common scoring template, for instance, a Lesion Visualization Grading Score. The scoring template was generated by prostate gland regions as illustrated in FIG. 14 and Tables 3 and 9, below. The scoring template was generated by pelvic lymph node regions as shown in FIG. 15.

TABLE 2

| Scoring | |
|---|---|
| Region | Region Names |
| RS | Right Seminal Vesicle |
| LS | Left Seminal Vesicle |
| RB | Right Base |
| LB | Left Base |
| RM | Right Mid |
| LM | Left mid |
| RA | Right Apex |
| LA | Left Apex |

The Lesion Visualization Grading Score in the location within the region corresponding to each individual area with suspected activity is numerically defined as follows:

TABLE 3

| Score | Description |
|---|---|
| 0 | Equal to Background activity/no |
| | contrast/no lesions observed |
| 1 | Slightly above background/poor contrast |
| 2 | Above background/good contrast |
| 3 | Above background/excellent contrast |
| 4 | Greater than all other activity/excellent |
| | contrast |

The reader scores may be converted into a binary measure (hi/lo or pos/neg). The primary endpoint was the ability of trofolastat (compound of Formula (1)) to detect prostate cancer within the gland. Secondary endpoints included detection of extent and location within the gland, pelvic lymph nodes and comparative performance against MRI.

Results.

87 patients were enrolled from 16 centers with interim data available for 54 subjects in the Phase 2 study. The patients had the following demographics and baseline characteristics as shown in Table 4 below:

TABLE 4
Demographics (n = 54)

| | N | % |
|---|---|---|
| Age | 63 (47-76) | |
| Race- White | 52 | 96.3 |
| Neoadjuvant Hormone Therapy | 15 | 27.8 |
| Clinical T Stage | | |
| ≦T1B | 0 | |
| T1C | 1 | 1.8 |
| T2 | 0 | |
| T2A | 7 | 12.9 |
| T2B | 14 | 25.9 |
| T2C | 10 | 18.5 |
| T3 | 1 | 1.8 |
| T3A | 15 | 27.8 |
| T3B | 6 | 11.1 |
| ≧T3C | 0 | |
| Baseline PSA | 12.8 ng/ml (2.7-137.1) | |

A majority (greater than 2 out of 3) of SPECT/CT readers correctly identified the presence or absence of primary prostate cancer in 51 out of 54 (94%, 85-98 CI) of the patients, including 2 true-negative cases. Sensitivity and specificity were 94% (84-98 CI) and 100% (34-100 CI), respectively.

TABLE 5

Pathology

| | | positive | negative |
|---|---|---|---|
| ^{99m}Tc-MIP-1404 scan | positive | 49 | 0 |
| (Formula 1 compound) | negative | 3 | 2 |

TABLE 6
^{99m}Tc-MIP-1404 SPECT/CT Performance Characteristics
Interim ^{99m}Tc-trofolastat SPECT/CT Performance Characteristics

| | Sensitivity | Specificity | Accuracy | PPV | NPV |
|---|---|---|---|---|---|
| Analysis Group | (95% CI) | (95% CI) | (95% CI) | (95% CI) | (95% CI) |
| Patient/Gland Level | 0.84 | 1.00 | 0.94 | 1.00 | 0.40 |
| (n = 54) | (0.84-0.98) | (0.34-1.00) | (0.85-0.88) | (0.83-1.00) | (0.12-0.78) |

^{99m}Tc-containing Formula (1) compound with SPECT/CT imaging accurately detects primary prostate carcinoma with high sensitivity and specificity in high-risk patients prior to surgery. The positive interim data for the Phase 2 trial of ^{99m}Tc-MIP-1404 as a diagnostic imaging agent met the primary endpoint of detecting prostate cancer within the gland, showing high sensitivity and specificity.

Three nuclear medicine experts and an MRI expert, blinded to clinical information, assessed ^{99m}Tc-trofolastat uptake and morphologic features respectively in the prostate gland and lymph nodes. The findings were recorded using common anatomic template consisting of six prostate segments and pelvic lymph node regions. Following the standard of care RP and ePLND surgery, step-section histopathologic evaluation was performed and Gleason Score (GS) for lesions in the prostate gland and an indication of positive or negative for PCa in lymph node regions were recorded by an on-site pathologist no more than 3 weeks after ^{99m}Tc-trofolastat dosing. For each analysis level (gland/patient. lobe, and lymph node regions), a result was considered positive if any positive finding (regardless of size) exists within the level, and negative if no positive findings. ^{99m}Tc-trofolastat and histopathology results of the prostate gland were evaluable for the first 54 patients and in lymph node regions for 53 patients. 1,981 nodes were sampled from 54 patients with a mean size of 3.9 mm for positive nodes. 16/53 (30%) patients had histopathologically confirmed lymph node involvement. The ^{99m}Tc-trofolastat scans detected primary prostate cancer that was confirmed by histopathologic findings in 51/54 (94%) evaluable patients and lymph node involvement in 16/53 (30%) patients in which 8/16 (50%) were confirmed by histopathology. Further, the method is able to detect masses of about 3.9 min and potentially smaller, as the 3.9 mm dimension is a mean of all positive nodes (range=0.2 to 16 mm). In an example, patient's positive by ^{99m}Tc-trofolastat scan with matching positive histopathology, the ^{99m}Tc-labeled PSMA inhibitor had sensitivity to detect positive lymph nodes 2 mm in size.

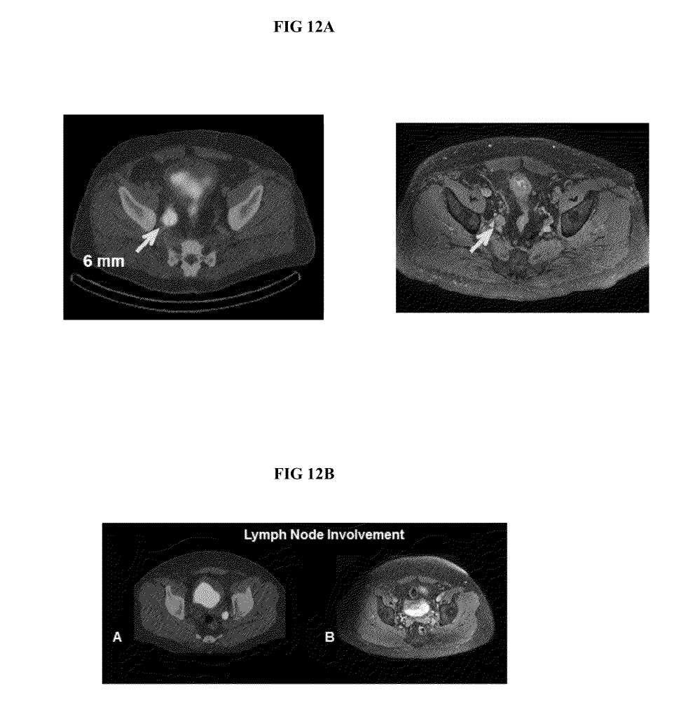

FIG. 12A compares fused axial ^{99m}Tc-MIP-1404 SPECT/CT reconstruction (left), and axial T1W MRI (right), different from that in FIG. 12B (below). The arrows indicate a histologically confirmed positive 6 mm right obturator lymph node read as positive by the ^{99m}Tc-MIP-1404 SPECT/CT reader and positive by the MR reader. FIG. 12B (A) indicates a histologically confirmed positive 5 mm left hypogastric lymph node read as positive by all ^{99m}Tc-trofolastat SPECT/CT reader and negative by the MR reader (FIG. 12B(B)).

### Example 5

Comparison of ^{99m}Tc-MIP-1404 SPECT/CT imaging with standard MRI for accurately detecting primary prostate cancer and lymph node metastasis.

Methods:

The methodology of Example 4 was used. Three nuclear medicine experts and an MRI expert, blinded to clinical information, assessed ^{99m}Tc-MIP-1404 uptake and morphologic features respectively in the prostate gland and lymph nodes. The assessments were made using a common scoring template, for instance, a Lesion Visualization Grading Score. The scoring template may be generated by regions as described in Table 2 above and illustrated in FIG. 14 for prostate gland scoring and in FIG. 15 for pelvic lymph node scoring. The Lesion Visualization Grading Score in the location within the region corresponding to each individual area with suspected activity is numerically defined as described in Table 3 above. These scores were compared to on-site histopathology results obtained subsequent to RP and EPLND.

Results:

87 patients were enrolled from 16 centers with interim data available for 54 subjects in the Phase 2 study. ^{99m}Tc-MIP-1404 SPECT/CT and histopathology results in the gland were evaluable for the first 54 patients and in 53 patients with lymph node involvement. MR images were evaluable in 47 of 54 patients. ^{99m}Tc-MIP-1404 SPECT/CT readers and MRI readers correctly characterized primary disease in 44/47 (94%) and 38/47 (81%) matched patients, respectively. ^{99m}Tc-MIP-1404 SPECT/CT readers correctly characterized primary prostate carcinoma in six (13%) more patients than the MRI reader, suggesting improved sensitivity and accuracy over MRI.

FIG. 7 compares examples of primary prostate lesions as seen in fused axial ^{99m}Tc-MIP-1404 SPECT/CT reconstructions from four study patients (row A), and matching axial T1W MRIs (row B), arranged by Gleason score from left to right. Red arrow heads indicate the location of histologically confirmed primary prostate lesions. In the first patient (far left) normal pathology as assessed by ^{99m}Tc-MIP-1404 SPECT/CT scoring (Row A) which was incorrectly read as a positive diagnosis by MRI (Row B), led to a potentially unnecessary prostatectomy. The superior accuracy of ^{99m}Tc-MIP-1404 SPECT/CT imaging compared to MRI can prevent unnecessary surgeries by enabling doctors and patients to make more informed treatment decisions.

TABLE 7
Matched ^{99m}Tc-MIP-1404 SPECT/CT vs. MRI Primary Disease Performance
Characteristics
Interim Matched ^{99m}Tc-trofolastat SPECT/CT vs. MRI Performance Characteristics

| Analysis Group | Sensitivity | Specificity | Accuracy | PPV | NPV |
|---|---|---|---|---|---|
| (n = 47) | (95% CI) | (95% CI) | (95% CI) | (95% CI) | (95% CI) |
| Patient/Gland - ^{99m}Tc-trofolastat | 0.93 | 1.00 | 0.94 | 1.00 | 0.40 |
| | (0.82-0.98) | (0.29-1.00) | (0.83-0.98) | (0.92-1.00) | (0.12-0.78) |
| Patient/Gland - MRI | 0.84 | — | 0.81 | 0.95 | — |
| | (0.71-0.92) | | (0.67-0.90) | (0.83-0.98) | |

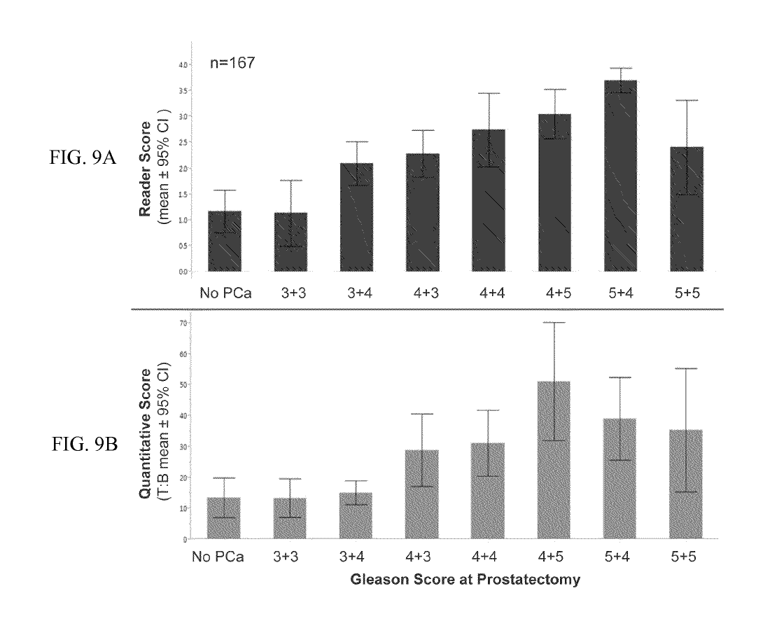

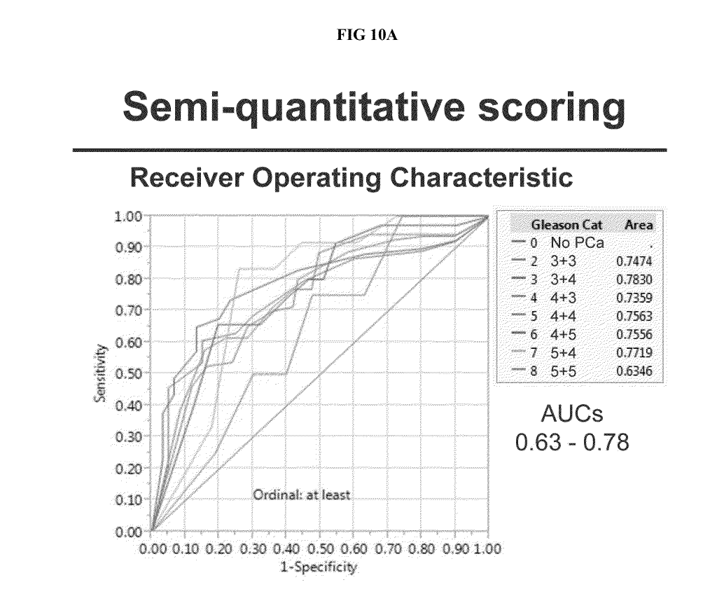

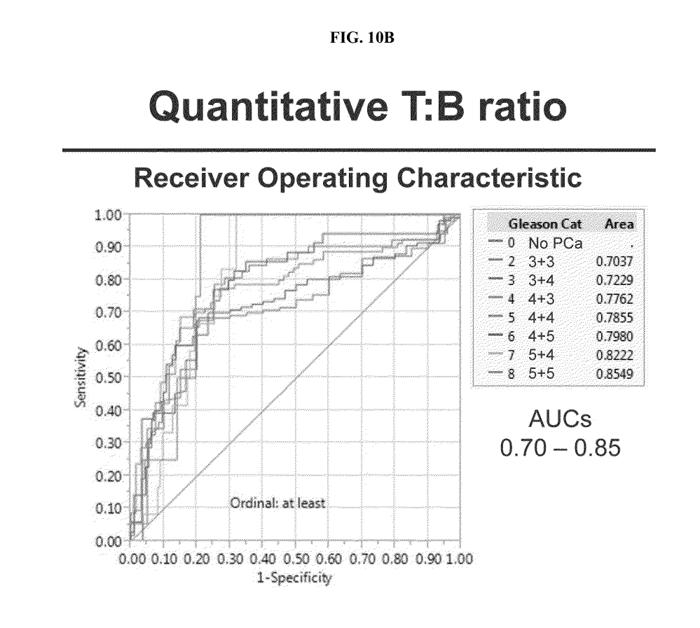

Data from the phase 2 trial was analyzed using a semi-quantitative (5-point scoring system; see Table 9 for approximate T:B ratio for each score) and a quantitative evaluation (T:B ratio=maximum count value: background mean value; each from a circular ROI with 2 cm diameter; see FIG. 8). Both quantitative and semi-quantitative methods of assessing prostate gland/lobe uptake of ^{99m}Tc-MIP-1404 show highly significant correlation with Gleason score (p<0.0001). See FIG. 9A (a reader score from a semi-quantitative measurement correlation with Gleason Score (p<0.0001); Spearman's ρ=0.476); 9B (a quantitative score, based upon T:B ratio, correlation with Gleason Score (p<0.0001); Spearman's ρ=0.504); 10A (semi-quantitative scoring showing that reader discriminate lobes with ≧3+3 and ≧3+4 from normal lobes better than quantitation alone), and 10B (quantitative T:B ratio showing better discrimination with quantitation in high grade disease from normal lobes than reader semi-quantitative scores). “Spearman's” refers to Spearman's Correlation Coefficient, a non-parametric statistical test. As used above, the quantitative maximum count value is the maximum counts of detected gamma photons, which is a unit-less measure.

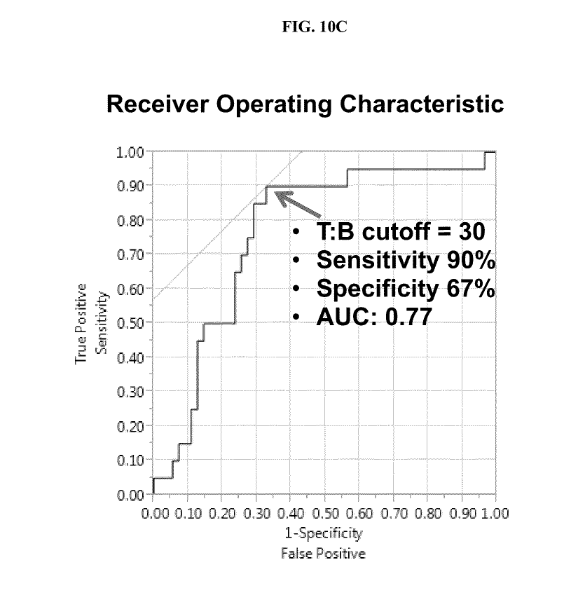

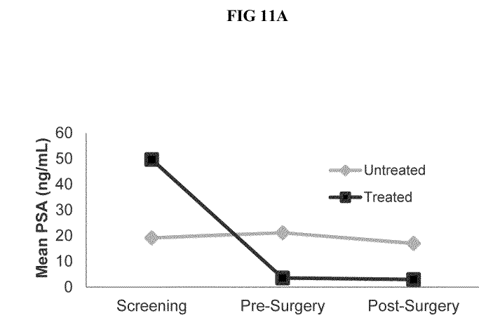

FIG. 10C describes the quantitative measure of ^{99m}Tc-MIP-1404 uptake (Tumor or Target:Background) as a predictor of metastatic lymph node involvement at the time of surgery. In the phase 2 clinical trial, patients were to undergo imaging with ^{99m}Tc-MIP-1404 prior to having radical prostatectomy with extended pelvic lymph node dissection. All resected lymph node tissue was assessed for prostate cancer by a site pathologist to determine if the patient was deemed to have metastatic prostate cancer in the local lymph nodes. A statistical analysis, ROC, was performed to generate a curve which plots the rate of true positives (y-axis) with the corresponding rate of false positives (x-axis) at differing cut-off points of the quantitative measure where a determination of lymph node involvement could be derived. This plot was used to determine the optimal point at which there is a maximization of the true positive rate (sensitivity) and a minimization of the false positive rate (1-specificity). This point was determined to be approximately at a target:background of 30 in the primary prostate tumor which yields a sensitivity of 90% and specificity of 67% for predicting lymph node involvement prior to surgery (90%; 18 out of 20 patients with lymph node involvement and no prior treatment had T:B value of ≧30 in the primary tumor. Additionally, the area under the curve can be calculated which corresponds to the diagnostic accuracy of the test over a range of values. Depending on the particular set of clinical circumstances, it may be more appropriate to select a point where specificity is maximized instead of sensitivity. The ROC curve allows for the performance of the test to be observed over the entire range of possibilities. ^{99m}Tc-MIP-1404 can predict, with a high degree of accuracy, which patients are likely to harbor metastatic disease based on a non-invasive measurement of the primary tumor.

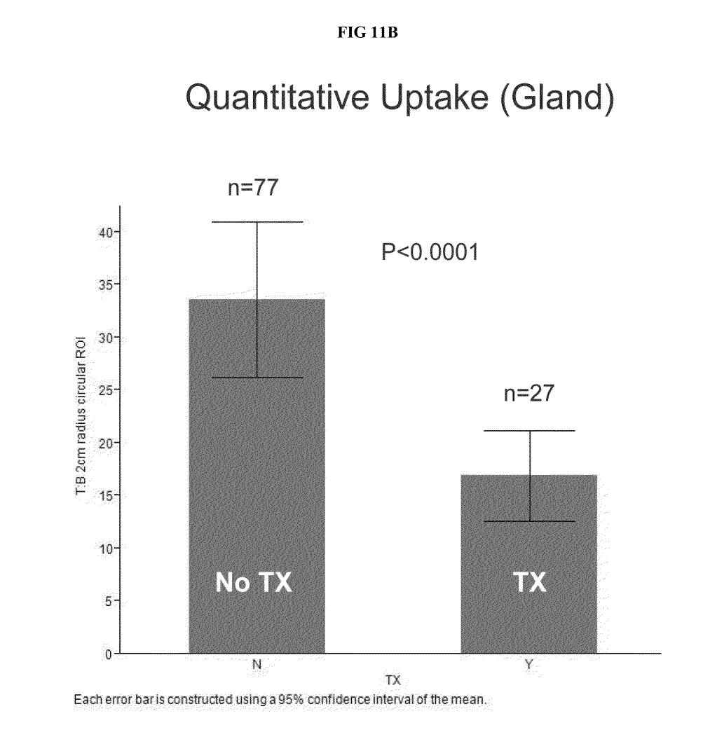

Effect of prior prostate cancer treatment on prostate gland/lobe uptake of ^{99m}Tc-trofolastat was also analyzed. Of patients who received prior prostate cancer treatment (neoadjuvant therapy), the majority of the patients received one or more doses of one or more hormonal therapies. The hormonal therapeutics included degarelix, goserelin, casodex (biculutamide), lupron, diphereline and leuproreline. Two prior-treated patients received enzalutamide (MDV3100), alone. One patient received an antimitotic chemotherapy (docetaxel) along with hormonal therapy. The results in FIGS. 11A (all treated patients vs. untreated patients) and 11B (Tx is prior treated patients, no Tx is patients who were not treated prior to imaging) indicate the SPECT/CT assay may be used to monitor efficacy of a prostate cancer treatment, as the uptake of ^{99m}Tc-MIP-1404 in the prostate gland was significantly lower in patients who had received treatment prior to assay compared to patients who had not received prior prostate cancer treatment (p<0.0001). The lower T:B ratios in these prior-treated patients also correlated with declining PSA levels, lending further evidence of efficacy of treatment. It is also to be noted from FIG. 11B, that the treated patients had a much lower uptake of ^{99m}Tc-MIP-1404. Accordingly, the level of uptake of the ^{99m}Tc-MIP-1404 may be directly correlated to disease progression and/or aggressiveness, as further shown below. As used above, neoadjuvant therapy refers to a primary treatment regimen.

The ^{99m}Tc-MIP-1404 SPECT/CT readers accurately characterized lymph node involvement in 77% ( 41/53) of patients and MRI readers accurately characterized lymph node involvement in 75% ( 35/47) of patients. FIG. 12B compares fused axial ^{99m}Tc-MIP-1404 SPECT/CT reconstruction (A), and axial T1W MRI (B) in a different patient than that presented in FIG. 12A. Red arrows indicate a histologically confirmed positive 5 mm left hypogastric lymph node read as positive by the ^{99m}Tc-MIP-1404 SPECT/CT reader and negative by the MR reader. ^{99m}Tc-MIP-1404 SPECT/CT imaging can correctly identify lymph node metastasis which is undetectable by MRI, leading to earlier diagnosis, more accurate prognosis, and more successful treatment.

### Example 6

Comparison of whole-body ^{99m}Tc-MIP-1404 SPECT/CT imaging with conventional bone scan for detecting suspected areas of bone metastasis.

Methods:

The methodology of Example 4 was used. Whole-body planar scintigraphic images using ^{99m}Tc-MIP-1404 were evaluated by 3 readers blinded to clinical information to determine if disease was present beyond the pelvic region. The ^{99m}Tc-MIP-1404 whole-body images were compared to the bone scan images.

Results:

The whole-body planar images using ^{99m}Tc-MIP-1404 show clear illumination of the prostate, lymph nodes, liver and kidneys in the image at 4 hours post administration. SPECT/CT images of patients at 4 and 24 hours demonstrated excellent lesion contrast with target to background ratios ranging from 3:1 to 28:1 at 4 and 24 hours respectively. ^{99m}Tc-MIP-1404 rapidly localized to lesions in lymph nodes and bone as visualized by whole-body imaging as early as 1 hour post-injection in men with prostate cancer.

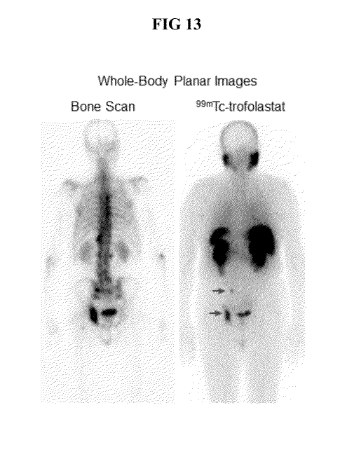

FIG. 13 illustrates the increased accuracy and specificity of a whole-body planar ^{99m}Tc-MIP-1404 scan versus conventional bone scan. The ^{99m}Tc-MIP-1404 scan (right) shows only PSMA expressing sites (arrows), consistent with skeletal metastases. Comparatively, the bone scan (left) displays multiple areas of non-specific uptake which can confound diagnosis of metastatic disease.

Detection of suspected tumor metastasis to the bone is evident earlier during the clinical course of the cancer with ^{99m}Tc-MIP-1404 as compared to other conventional radionuclide imaging agents used in the clinic. Because imaging with ^{99m}Tc-MIP-1404 permits early detection of tumor and metastasis, early therapeutic interventions may be possible to stem the progression and spread of prostate cancer.

Moreover, two men in the Phase 2 trial who, per protocol, had undergone prostatectomy were shown to have suspected metastatic disease in the bone using ^{99m}Tc-MIP-1404. Clinical care protocol in prostate cancer today recommends that if metastatic disease has reached bone, prostatectomy is contraindicated and systemic treatment recommended (e.g. chemotherapy). Thus ^{99m}Tc-MIP-1404 imaging provides early detection of metastases as well as primary disease, quickly and accurately guiding clinicians to appropriate diagnosis, prognosis, and therapy, and therein preventing needless biopsies or unwarranted radical prostatectomies.

### Example 7

### Methods

The suitability of a compound represented by Formula (1) to detect and discriminate between tumor tissue and normal tissue was tested in a Phase 1 study by comparing the SPECT/CT images obtained using a compound represented by Formula (1) with step-section histopathology in 8 patients (pts) undergoing radical prostatectomy. Briefly, 8 patients were administered 20mCi of the Formula (1) compound and SPECT/CT images of the pelvis were acquired 2 hours after injection. The T/B ratio was calculated for six segments of the prostate gland based on the images. Imaging results in segments and right and left lobes of the prostate were compared with Primary Gleason Grades (PGG) and total Gleason Scores (GS) recorded by a blinded pathologist. Sensitivity, specificity, accuracy for a T/B threshold of 5.9 were calculated by a receiver operator curve as explained above.

Results:

SPECT/CT imaging using the compound represented by Formula (1) correctly identified the presence of primary prostate cancer in all patients participating in the study. Imaging discriminated high-grade prostate cancer (GS≧7) from moderate and low-grade (GS<7) or no disease with an accuracy of 93.8% in lobes and 81.3% in segments. Accuracy increased to 89.6% in segments with dominant primary lesions with PGG <4 or ≧4 (see table 8).

TABLE 8

| | Lobe Gleason | Segment Gleason | Segment Dominant |
|---|---|---|---|
| | Score ≧7 | Score ≧7 | Grade ≧4 |
| Sensitivity (%) | 92.3 | 71.4 | 90.0 |
| Specificity (%) | 100 | 95 | 89.3 |
| Accuracy (%) | 93.8 (15/18) | 81.3 (39/48) | 89.6 (43/48) |
| AUC ± SE | 0.969 ± 0.04 | 0.87 ± 0.05 | 0.942 ± 0.04 |

At a T/B threshold ratio of 5.9, SPECT/CT imaging with the Formula (1) compound accurately characterized segments of the prostate gland with moderate or low-grade disease and accurately discriminates no disease patients from those containing higher-grade disease. The results above indicates that imaging with the compound of Formula (1) can provide prognostic information for both local and distant disease in a single scan, thus permitting a clinician to make a decision about treatment based on the images from a single scan.

### Example 8

### Scoring and Analysis of ^{99m}Tc-MIP-1404 SPECT/CT Images

Three SPECT/CT readers conducted impartial and independent assessments of ^{99m}Tc-MIP-1404 SPECT/CT and planar imaging data for each patient. The SPECT readers assessed reconstructed SPECT/CT data and assigned Lesion Visualization Grading Scores (Table 3), by region, for both the prostate and pelvic lymph nodes. Planar images were assessed to determine whether disease was evident outside of the prostate. Each of the 3 SPECT/CT readers assessed each case independently and made their own final determinations.

The SPECT/CT Assessment included the following:
Only 1 time point (post-study-drug injection), including whole body planar and SPECT/CT of the pelvis images, were assessed by each SPECT/CT reader. SPECT/CT readers evaluated the SPECT image dataset using the concomitant CT portion of the exam for anatomical reference.
Each image data assessment consisted of an evaluation of 6 regions within the prostate gland as well as an assessment of the pelvic lymph nodes (See FIGS. 14 and 15). SPECT/CT Reviewers evaluated each defined region and applied a grading score ranging from 0 to 4 (See Table 3).
SPECT/CT readers qualitatively determined if disease was present beyond the pelvic region and if the subject was positive or negative for prostate cancer.
For each analysis level (subject, gland, and region), a result was considered positive for prostate cancer if any positive finding existed within the level, and negative if there were no positive findings. For example, for a given subject, if only 1 of 6 regions of the prostate gland was positive, then the gland-level was positive. However, on the regional-level for the same subject, only the 1 region within the gland that had the positive finding was considered positive, and all other regions of the gland were considered negative.
For all analyses, any ^{99m}Tc-MIP-1404 scans that were unreadable were considered not evaluable (NE).

Image Technical Quality Assessment:

SPECT/CT readers began each image data review by ensuring that all images displayed for assessment were recorded by modality and anatomical coverage (i.e., whole-body planar and SPECT/CT-pelvis). SPECT/CT readers then rated the overall quality of the image data. Three general quality categories were applied: Optimal, Readable but Not Optimal, and Not Readable.

If a SPECT/CT reader selected Optimal or Readable but Not Optimal for either a SPECT or whole-body image, assessments were begun. If a SPECT/CT reader described the overall image quality as Not Readable, no assessments were entered.

SPECT image reconstruction was performed using an iterative OSEM (Ordered-Subset Expectation Maximization) technique and corrected for attenuation using an Oasis imaging workstation (Segami Corp., Columbia, Md., USA) or equivalent imaging workstation. The derivation of the iterative OSEM algorithm and analysis as applied to SPECT has been previously described (Hudson et al., IEEE Trans. Med. Imag., (1994), p 100-108).

Quantitative use of ^{99M}Tc SPECT/CT taking into account the nonstationary behavior of OSEM reconstruction when used in the clinical operation range has also been described (Zeintl et al., J. Nucl. Med. (2010), p 921-928). Current commercially available SPECT/CT technology using OSEM-3D reconstruction, CT-based attenuation correction, and scatter correction allows quantification of ^{99m}Tc radioactivity concentration in absolute terms.

Pelvic Lymph Node Assessment:

SPECT/CT readers were presented with whole-body images followed by the axial, coronal, and sagittal reconstructed slices with attenuation correction, color scale, and intensity. SPECT/CT readers evaluated the ^{99m}Tc-MIP-1404 whole-body planar images in addition to the SPECT/CT to determine whether there was disease present outside of the prostate gland and lymph nodes. If the determination was positive, the readers recorded a comment stating the location of the disease.

SPECT/CT readers then entered a Lesion Visualization Grading Score (See Table 3 above) for pelvic lymph node scoring for each region corresponding to a grouping of lymph nodes (right and left sides) (See FIG. 15).

Lymph nodes with activity or uptake of ^{99m}Tc-MIP-1404 greater than that of normal lymph nodes and the immediate background were considered positive. Inguinal nodes were useful as a visual reference to evaluate normal activity.

Prostate Gland/Seminal Vessel Assessment: SPECT/CT readers were presented with fused axial slices in a 4×2 format, (with color and intensity displays; see FIGS. 3B and 3C). The images were centered over the prostate gland and the SPECT/CT readers were required to enter a Lesion Visualization Grading Score (See Table 9) for each of the 6 defined prostate regions plus 2 seminal vesicle regions (See Tables 2 and 3, above, and FIG. 14).

SPECT/CT readers assessed each anatomic location on axial, coronal, and sagittal SPECT/CT image data corresponding to the 6 prostate regions and 2 seminal vesicles to determine if there was any area suspicious for prostate cancer. In healthy volunteers, the normal prostate was expected to have uptake within a target to background range of 4:1 to 6:1 where the background is taken from normal tissue within normal muscle in the pelvis.

TABLE 9
Prostate Scoring Scale - Semi-Quantitative Evaluation:

| | | Target:Background |
|---|---|---|
| | | Ratio (approximate |
| Score | Description | values) |
| 0 | Equal to Background activity/no | ≦6 |
| | contrast/no lesions observed | |
| 1 | Slightly above background/poor | >6 and ≦8 |
| | contrast | |
| 2 | Above background/good contrast | >8 and ≦10 |
| 3 | Above background/excellent contrast | >10 and ≦15 |
| 4 | Greater than all other activity/excellent | >15 |
| | contrast | |

### Example 9

Nomograms are developed to assess the probability of lymph node involvement (LNI) during a prostate cancer condition. Typically, nomograms consist of three to four variables. The present inventors will use a cohort of patients treated with RP including lymph node dissection (LND) to develop the nomogram. In one aspect, the three-variable nomogram will include basic clinical variables, such as pretreatment PSA, clinical stage, and biopsy Gleason grade. The four-variable nomogram may include the T/B ratio or may account for institutional with respect to the extent of the LND and pathologic evaluation of specimens.

Methods:

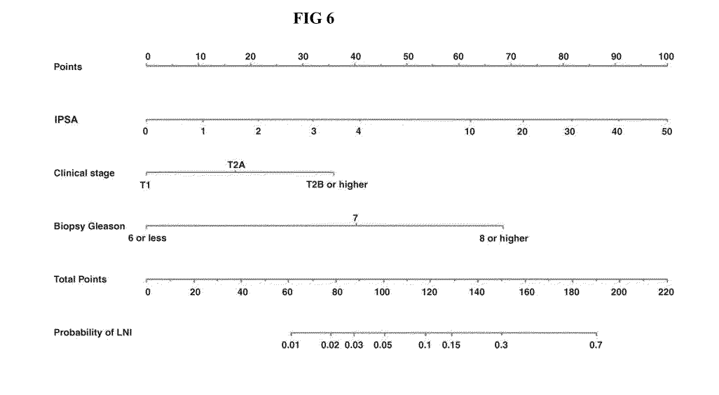

For each patient a pretreatment prostate-specific antigen (PSMA) score will be obtained and correlated to a numerical value on the initial PSMA (IPSA) axis. See FIG. 6. A straight line will be drawn from the IPSA axis to the Point's axis to determine how many points are to be assigned to evaluate the probability of a positive LNI. This process will be repeated for each variable in the nomogram. The final sum of the points for each of the variables in the nomogram will be calculated. After locating the final sum on the Total Points Axis the patient's probability of having positive lymph node involvement will be estimated using the probability of LNI axis.

The decision to pursue or not to pursue a specific therapeutic protocol is challenging. Both the Gleason score and clinical stage of a prostate cancer are considered prior to starting a specific therapeutic protocol. As mentioned above, Table 1 correlates the T/B ratio to the Gleason score. A correlation also exists between the stage of a prostate cancer and the Gleason score. For example, Godoy et al., disclose that patients with stage T1 prostate cancer have a Gleason score ≦6.0. Patients with stage T2a prostate cancer had Gleason scores of about 7.0, while patients with stage T2b prostate cancer had Gleason scores ≧8. The Gleason scores for patient with stage T3 prostate cancer is about 9.0. Using the correlation between Gleason score and the stage of a prostate cancer condition and the correlation between the T/B ratio and Gleason score it will be possible to evaluate the status of a patient with prostate cancer condition.

### Example 10

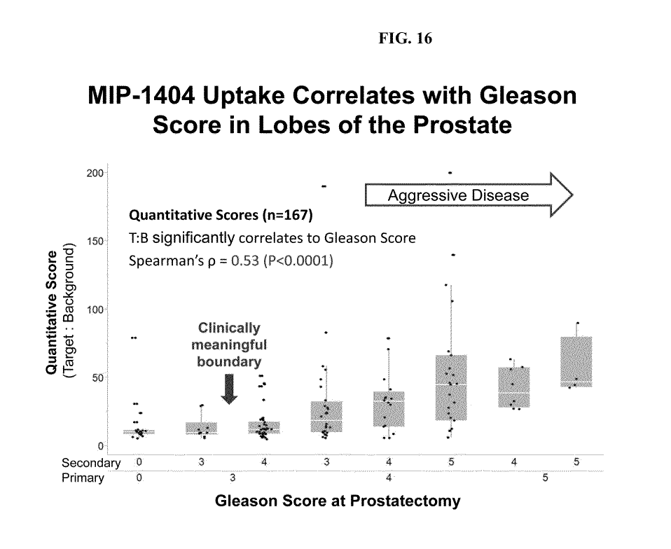

^{99m}Tc-MIP-1404 Uptake Correlation with Gleason Score in Lobes of the Prostate. FIG. 16 is a graph describing the relationship between the quantitative measure of ^{99m}Tc-MIP-1404 uptake (Target:Background) in lobes of the prostate and the histopathologic assessment following radical prostatectomy in the phase 2 clinical trial. A total of 167 lobes were evaluable with both a SPECT/CT scan and pathology results. Non-parametric statistical tests for correlation (Spearman's correlation coefficient or rho) of the quantitative measures with categorized Gleason scores were calculated. The values were found to significantly correlate, and are likely non-random (P<0.0001). This demonstrates that there exists a positive, statistically significant correlation between ^{99m}Tc-MIP-1404 uptake and Gleason score. The relationship shows that ^{99m}Tc-MIP-1404 uptake is useful as a non-invasive surrogate measure of disease aggressiveness.

### Example 11

It is expected that ^{99m}Tc-MIP-1405 will behave similarly to ^{99m}Tc-MIP-1404. Accordingly, if the above experiments were to be conducted with ^{99m}Tc-MIP-1405, similar results would be obtained, exhibiting albeit potentially different absolute numbers, but similar trends and methods would be observed.

### Example 12

A phase 3 study plan for ^{99m}Tc-trofolastat chloride is provided below. The title of the study is: MIP-1404 3301/A Phase 3 Study to Evaluate the Safety and Efficacy of ⁹⁹¹Tc-MIP-1404 SPECT/CT Imaging to Detect Clinically Significant Prostate Cancer in Men with Biopsy Proven Low-Grade Prostate Cancer who are Candidates for Active Surveillance. The indication is for the use of ^{99m}Tc-trofolastat chloride, a radioactive diagnostic agent, for single-photon emission computed tomography imaging of the prostate gland indicated in men with biopsy-confirmed prostate cancer as an aid to identify clinically-significant prostate cancer. In some embodiments, the use of ^{99m}Tc-trofolastat chloride may be indicated in men suspected of having prostate cancer, but for which no surgical or biopsy procedures have been conducted. In still another embodiment ^{99m}Tc-trofolastat chloride is indicated for imaging newly diagnosed patients with prostate cancer whose biopsy indicates a histopathological Gleason grade of <or equal to 3+4 severity and who are candidates for active surveillance as well as prostatectomy. In these patients, the ^{99m}Tc-trofolastat chloride imaging results may be used to help estimate the risk for detecting a histopathological Gleason grade of 3+4 or higher at prostatectomy. Approximately 300 patients will be enrolled, and the ^{99m}Tc-trofolastat chloride (i.e. MIP-1404) will be administered as a single intravenous injection. The study objectives are fourfold: 1. To evaluate the safety and tolerability of MIP-1404 in subjects with biopsy proven low-grade prostate cancer; 2. Sensitivity of three blinded MIP-1404 SPECT/CT readers (⅔ readers succeeding at least 70%; with a lower Confidence interval of 60%) to identify subjects with clinically-significant prostate cancer (Gleason score >3+4) at radical prostatectomy; 3. Specificity of three blinded MIP-1404 SPECT/CT readers (⅔ readers succeeding at least 70%; with a lower Confidence interval of 60%) to identify subjects without clinically-significant prostate cancer (Gleason score <3+4) at radical prostatectomy (RP); and 4. To determine the area under the receiver operating characteristic curve AUC_{ROC} (true positive rate vs false positive rate) of SPECT/CT imaging of the prostate using MIP-1404 to discriminate clinically significant prostate cancer (Gleason score >3+4, >0.5 cc volume) in subjects eligible for active surveillance. Clinically significant cancer is an art-recognized term (Epstein et al. J. Am. Med. Assoc. 271(5):368-74 (1994).

The study design includes a multicenter, multi-reader, open-label trial, comparing MIP-1404 SPECT/CT imaging in newly diagnosed men who have had a diagnostic trans-rectal ultrasound (TRUS) guided biopsy with a histopathologic finding of Gleason score ≦3+4 (no dominant pattern 4) and who are eligible for active surveillance, but have decided to have radical prostatectomy with or without a pelvic lymph node dissection. This study will evaluate the diagnostic accuracy of MIP-1404 SPECT/CT assessments by three readers blinded to clinical information, in correctly identifying subjects with previously unknown clinically-significant prostate cancer (Gleason score >3+4) using the whole-mounted step-sectioned histopathologic assessment of the prostate gland following radical prostatectomy as the truth standard. Subjects will receive a single IV dose of MIP-1404 (study drug) followed by SPECT/CT scan 3-6 hours after injection. Subjects will have elected to undergo a standard of care RP surgery and histological assessment of specimens within four weeks after study drug dosing. MIP-1404 image data will be evaluated for visible uptake and compared with a central histopathology assessment for the presence or absence of clinically-significant prostate cancer.

The study population is for men with biopsy proven low-grade prostate cancer (Gleason score 3+3 or 3+4) who are candidates for active surveillance, but elect to have radical prostatectomy.

Inclusion criteria. Subjects must meet all of the following criteria to be enrolled in this study: 1. Male 18 years of age or older; 2. Ability to provide signed informed consent and willingness to comply with protocol requirements; 3. Diagnostic trans-rectal ultrasound (TRUS)-guided biopsy (10-12 cores) within 6 months of enrollment showing adenocarcinoma of the prostate gland with a Gleason score 3+3 or 3+4; 4. PSA<15.0 ng/mL (ug/L); 5. Scheduled to undergo radical prostatectomy with or without lymph node dissection; 6. Agreed to use an acceptable form of birth control for a period of 7 days after the MIP-1404 injection; 7. Subject has a life expectancy of >5 years; and ECOG Performance Status 0, 1 or 2.

Exclusion criteria. Subjects who meet any of the following criteria will be excluded from the study: 1. Subjects not eligible for active surveillance according to guidelines at clinical study site; 2. Subjects administered a radioisotope within 5 physical half-lives prior to study drug injection; 3. Previous treatment of prostate cancer or BPH including hormonal therapy, surgery (except prostate biopsy), radiation therapy, LHRH analogs, and non-steroidal anti-androgens or any 5α-reductase inhibitors; 4. Planned androgen or anti-androgen therapy prior to surgery; 5. Subjects with any medical condition or other circumstances that, in the opinion of the investigator, would have significantly decreased obtaining reliable data, achieving study objectives, or completing the study; 6. Malignancy (not including curatively treated basal or squamous cell carcinoma of the skin) within the previous 5 years. (Ta bladder cancer with negative surveillance cystoscopy within the past 2 years may be included.).

Duration. The duration of subject participation will be from the time of signing informed consent through day following injection with MIP-1404 and completion of surgery.

Safety Assessments. Safety assessments will include monitoring of treatment-emergent adverse events, vital sign measurements and clinical safety laboratory values.

Statistical Methods. Approximately 265 subjects will be treated. Subject enrollment will continue until target enrollment has been reached and at least 100 patients having a rising PSA as defined by The Prostate Cancer Clinical Trials Working Group 2 (PCWG2) (a rising PSA that is greater than 2 ng/mL higher than the nadir; the rise has to be at least 25% over nadir and the rise has to be confirmed by a second PSA at least three weeks later) have been enrolled. The sample size provides 90% power at the alpha=0.025 one-sided level of significance that the AUC of the rater score-histopathology ROC curve will be equivalent or superior to the AUC under the null hypothesis with an equivalence limit difference of 0.1. The expected AUC for MIP-1404 treatment is assumed to be ≧0.7, and under the null hypothesis, the AUC is on the order of 0.5. All subjects who sign an informed consent document will be included in the enrolled subject population. All subjects who receive a dose of MIP-1404 will be included in the safety population. All subjects who receive a dose of MIP-1404, who undergo imaging and have histology results from prostatectomy will be included in the evaluable population. AE incidence, severity, and causality will be summarized using the Medical Dictionary for Regulatory Activities (MedDRA) preferred term and system organ class. Serious adverse events will be tabulated separately. Concomitant medication use will be tabulated. Changes from baseline vital signs and clinical laboratory parameters will be summarized by scheduled assessment.

For the primary endpoint, the mean of the maximum reader rating score will be analyzed against pathology results (Gleason score 3+3 vs >grade 3+) using logistic regression. The ROC curve, its AUC and confidence interval will be calculated from the logistic fit. The sensitivity and specificity of MIP-1404 to identify clinically-significant (Gleason score >3+4) prostate cancer based on histology as the gold standard will be calculated using cross-tabulation methods.

As an alternative statement for indications, other studies may be conducted. In one such alternative, it may be stated that MIP-1404 is a radioactive diagnostic agent for single-photon emission tomography imaging of the prostate gland indicated in men with biopsy-confirmed prostate cancer who have a Gleason score of less than or equal to 3+4 to assist clinicians in determining a patient's risk for more aggressive disease.

The following paragraphs provide additional embodiments:

### Embodiment 1

A method of identifying a severity level of prostate cancer in a patient clinically diagnosed with prostate cancer, the method comprising:
administering to the patient an effective amount of a compound that is ^{99m}Tc-trofolastat chloride;
acquiring an image of the patient;
determining a level of uptake of the compound in the prostate of the patient as a tumor (T) level;
determining a level of uptake of the compound in a control tissue as a baseline (B) level; and
assigning a severity level in terms a ratio of T:B below, at, or above a predetermined threshold value.

### Embodiment 2

The method of Embodiment 1, wherein the method is a non-surgical method.

### Embodiment 3

The method of Embodiment 1 or 2, wherein when the clinical diagnosis of prostate cancer is determined using a PSA value, digital rectal examination, trans-rectal ultra sound, symptomology, or a combination of any two or more thereof

### Embodiment 4

The method of Embodiment 1, 2, or 3, wherein when the clinical diagnosis of prostate cancer is determined using a PSA value, and the PSA value is <15.0 ng/ml.

### Embodiment 5

The method of any one of Embodiments 1-4, wherein a T:B ratio of ≦5.9 identifies the patient without clinically-significant prostate cancer at the time of the image acquisition.

### Embodiment 6

The method of any one of Embodiments 2-5, wherein the ratio of about ≦5.9 indicates low-grade prostate cancer or the absence of prostate cancer at the time of the image acquisition.

### Embodiment 7

The method of any one of Embodiments 2-6, wherein the patient is a candidate for active surveillance.

### Embodiment 8

The method of any one of Embodiments 2-7, wherein a T:B ratio of ≦5.9 is consistent with a Gleason score of ≦3+3.

### Embodiment 9

The method of any one of Embodiments 2-8, wherein a T:B ratio of ≦5.9 is consistent with a Gleason score of ≦3+4.

### Embodiment 10

The method of any one of Embodiments 1-9, wherein when the threshold value of greater than about 5.9 is highly sensitive for identifying the patient with clinically-significant prostate cancer at the time of the image acquisition.

### Embodiment 11

The method of any one of Embodiments 1-10, wherein a T:B ratio of >5.9 is consistent with a Gleason score of >3+4.

### Embodiment 12

The method of any one of Embodiments 1-10, wherein a T:B ratio of >15 is highly specific for identifying the patient with clinically-significant prostate cancer at the time of image acquisition.

### Embodiment 13

The method of Embodiment 11 or 12, wherein the patient is a candidate for cancer treatment.

### Embodiment 14

The method of Embodiment 13, wherein the treatment is hormonal, prostatectomy, radiation, LHRH (luteinizing hormone releasing hormone) analog, a non-steroidal anti-androgen, 5α-reductase inhibitor, antibody drug conjugate, or a combination of any two or more thereof

### Embodiment 15

The method of any one of Embodiment 1-14, wherein the determining comprises obtaining the image of the patient using nuclear medicine tomographic imaging techniques.

### Embodiment 16

The method of any one of Embodiment 1-15, wherein the patient has not received a prior prostate cancer treatment.

### Embodiment 17

A method for confirming tumor metastasis in a prostate cancer patient, the method comprising:
administering to the patient an effective amount of a compound that selectively binds to prostate-specific membrane antigen (PSMA), the compound represented by Formula 1 or Formula 2 or a pharmaceutically acceptable salt thereof;
imaging a region of interest in the patient;
obtaining a level of uptake of the compound by the prostate of the prostate cancer patient as a target (T) level;
obtaining a level of uptake of the compound in control tissue (B);
obtaining a quantitative score as a ratio of T:B; and
confirming metastasis if it is determined that the quantitative score is at, or above, a predetermined threshold value;
wherein: Formula I and Formula 2 are:

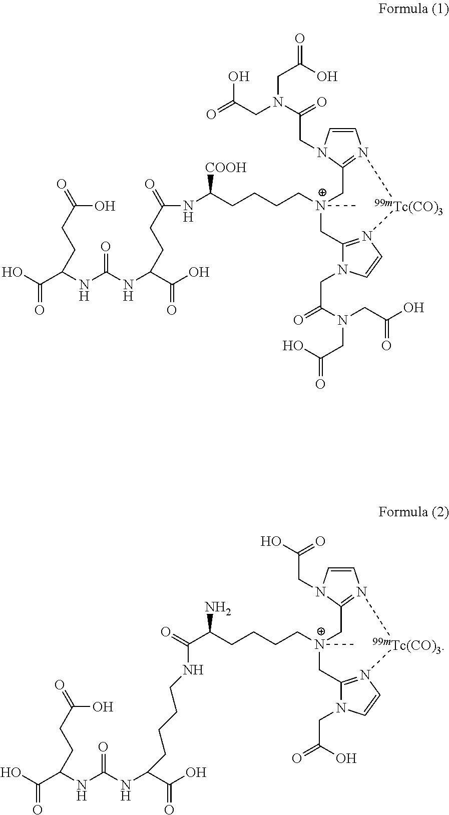

### Embodiment 18

The method of Embodiment 17, in which the predetermined threshold is chosen statistically to minimize undesirable effects of false positives and false negatives.

### Embodiment 19

The method of Embodiment 17 or 18, wherein the predetermined threshold is about 30.

### Embodiment 20

The method of any one of Embodiments 17-19 in which the patient is administered an effective amount of a compound of Formula 1.

### Embodiment 21

The method of any one of Embodiments 17-20 in which the imaging is performed using a nuclear medicine tomographic imaging technique.

### Embodiment 22

The method of Embodiment 21 in which the nuclear medicine tomographic imaging technique is selected from two-dimensional planar imaging, single-photon emission computed tomography (SPECT), or single-photon emission computed tomography combined with conventional computed tomography (SPECT/CT).

### Embodiment 23

The method of any one of Embodiments 17-22 in which the control tissue is normal prostate tissue, normal pelvic muscle, or normal pelvic lymph node.

### Embodiment 24

The method of any one of Embodiments 17-23, wherein the threshold value is a surrogate marker for aggressive prostate disease.

### Embodiment 25

The method of any one of Embodiments 17-23, wherein the threshold value is a surrogate marker for prostate metastasis.

### Embodiment 26

The method of any one of Embodiments 17-23, wherein the threshold value is a surrogate marker for a Gleason score of 7 or greater.

### Embodiment 27

A method for confirming lymph node involvement in a metastatic prostate cancer in a subject, the method comprising:
administering to the patient an effective amount of a compound that selectively binds to prostate-specific membrane antigen (PSMA), the compound represented by Formula 1 or Formula 2 or a pharmaceutically acceptable salt thereof;
determining a level of uptake of the compound in the prostate of the patient as a target (T) level;
determining a level of uptake of the compound in control tissue as a baseline (B) level; and
confirming lymph node involvement if a ratio of T:B is at, or above, a predetermined threshold value;
wherein: Formula I and Formula 2 are:

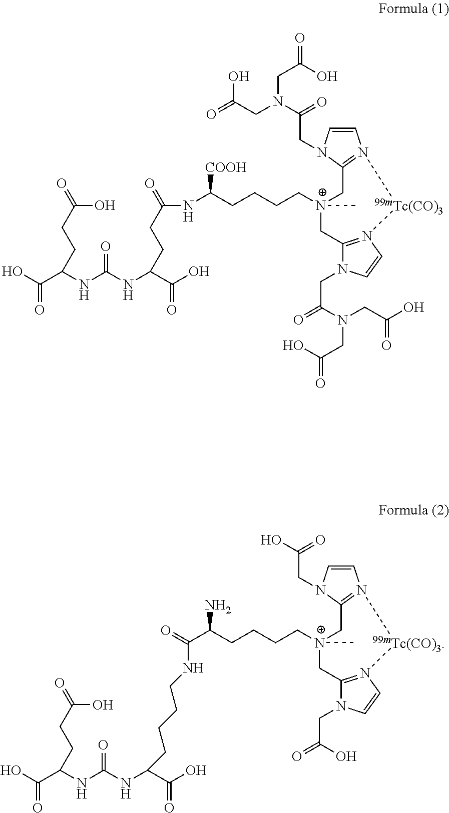

### Embodiment 28

The method of Embodiment 27, in which the predetermined threshold is chosen statistically to minimize undesirable effects of false positives and false negatives.

### Embodiment 29

The method of Embodiment 27 or 28, wherein the predetermined threshold is about 30.

### Embodiment 30

The method of any one of Embodiments 27-29 in which the compound of Formula (1) is administered.

### Embodiment 31

A kit comprising a first container including a free ligand MIP-1404, a second container including a ^{99m}Tc radionuclide, and instructions for producing ^{99m}Tc-trofolastat for: identifying a severity level of prostate cancer in a patient, confirming lymph node involvement in metastatic prostate cancer, confirming tumor metastasis, monitoring a status of prostate cancer, obtaining a SPECT/CT image of tissue expressing prostate-specific membrane antigen (PSMA) in vivo, detecting tumor metastasis to at least a portion of a bone or a soft tissue of a prostate cancer patient, identifying prostate tumor metastasis to a lymph node, monitoring the efficacy of prostate cancer treatment, monitoring or assessing a status of prostate cancer in a human subject, a non-invasive method of assessing a degree of disease aggressiveness in a human subject diagnosed with prostate cancer, assessing a likelihood of a presence of metastatic disease in a human subject diagnosed with prostate cancer, diagnosing metastatic disease in a patient clinically diagnosed as having prostate cancer, or identifying a severity level of prostate cancer in a patient harboring biopsy-confirmed prostate cancer.

### Embodiment 32

A kit comprising a radioactive diagnostic agent for nuclear medicine tomographic imaging of the prostate and instructions for diagnosing clinically-significant prostate cancer based upon a tumor:background (T:B) ratio that is below or equal to, or above a predetermined threshold value.

### Embodiment 33

The kit of Embodiment 32, wherein the instructions provide a T:B threshold value ≦5.9 indicative of clinically-nonsignificant prostate cancer.

### Embodiment 34

The kit of Embodiment 32 or 33, wherein the instructions provide a T:B threshold value >5.9 as highly sensitive for being indicative of clinically-significant prostate cancer.

### Embodiment 35

The kit of Embodiment 32, 33, or 34, wherein the instructions provide a T:B threshold value >15 as highly sensitive for being indicative of clinically-significant prostate cancer.

### Embodiment 36

The kit of Embodiment 32, 33, 34, or 35, wherein the instructions provide a T:B threshold value >30 as highly sensitive for being indicative of metastatic disease.

### Embodiment 37

A method of evaluating a human subject suspected of harboring a prostrate tumor, the method comprising:
administering to a human subject an effective amount of a gamma-emitting transition metal complex conjugated to a targeting moiety that selectively binds to prostate-specific membrane antigen (PSMA), including PSMA expressed on the surface of a prostate tumor;
subjecting the human subject to a nuclear medicine tomographic imaging technique to obtain one or more images of at least a portion of prostate tissue suspected of harboring tumor lesions;
assessing a level of uptake of said gamma-emitting transition metal complex conjugated to a targeting moiety by said at least a portion of prostate tissue compared to a level of uptake by control tissue; and
determining if a ratio of the level of uptake by said at least a portion of prostate tissue to the level of uptake by control tissue is below, at, or above a predetermined threshold.

### Embodiment 38

The method of Embodiment 37 in which the predetermined threshold is chosen statistically to minimize undesirable effects of false positives and false negatives.

### Embodiment 39

The method of Embodiment 37 or 38 in which the predetermined threshold is selected from the group consisting of 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9 and 7.0.

### Embodiment 40

The method of Embodiment 39 in which the predetermined threshold is 5.9.

### Embodiment 41

The method of any one of Embodiments 37-40 in which the evaluation is conducted non-invasively.

### Embodiment 42

The method of any one of Embodiments 37-41 in which the nuclear medicine tomographic imaging technique comprises two-dimensional planar imaging, single-photon emission computed tomography (SPECT), or single-photon emission computed tomography combined with conventional computed tomography (SPECT/CT).

### Embodiment 43

The method of any one of Embodiments 37-42 in which control tissue is elected from non-tumorous portions of prostate tissue or pelvic muscle tissue.

### Embodiment 44

The method of any one of Embodiments 37-43 further comprising subjecting the human subject to radical prostatectomy, cryosurgery, radiation therapy, hormone (androgen) deprivation therapy, chemotherapy, PSMA antibody-drug conjugate, or combinations thereof if it is determined that the ratio is at or above 5.9.

### Embodiment 45

The method of any one of Embodiments 37-44 further comprising electing not to subject the human subject to radical prostatectomy, cryosurgery, radiation therapy, hormone (androgen) deprivation therapy, chemotherapy, PSMA antibody-drug conjugate, or combinations thereof if it is determined that the ratio is below 5.9.

### Embodiment 46

The method of any one of Embodiments 37-45 further comprising subjecting the human subject to active surveillance monitoring if it is determined that the ratio is below 5.9.

### Embodiment 47

The method of any one of Embodiments 37-46 in which the human subject is reevaluated periodically.

### Embodiment 48

The method of any one of Embodiments 37-47 further comprising subjecting the human subject to watchful waiting monitoring if it is determined that the ratio is below 5.9.

### Embodiment 49

The method of Embodiment 48 in which changes in the human subject's symptoms are monitored.

### Embodiment 50

The method of any one of Embodiments 37-49 further comprising the detection of tumor lesions in a tissue other than prostate tissue.

### Embodiment 51

The method of any one of Embodiments 37-50 in which the transition metal is technetium-99m.

### Embodiment 52

The method of any one of Embodiments 37-51 in which the gamma-emitting transition metal complex conjugated to a targeting moiety comprises a compound represented by Formula (1):

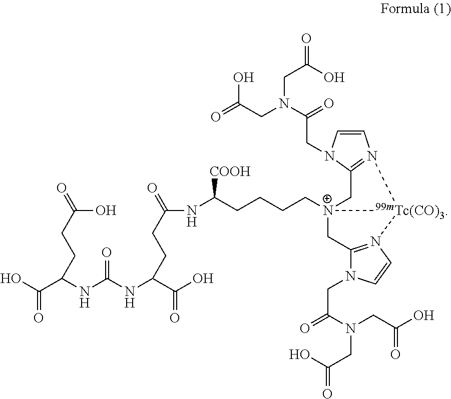

### Embodiment 53

The method of any one of Embodiments 37-52, wherein the gamma-emitting transition metal complex conjugates to a targeting moiety comprising Tc-trofolastat chloride.

### Embodiment 54

The method of any one of Embodiments 37-53 which is repeated periodically.

### Embodiment 55

The method of any one of Embodiments 37-54 which suggests that the human subject is harboring prostate cancer tumor if it is determined that the ratio is at or above 5.9.

### Embodiment 56

The method of any one of Embodiments 37-55 which suggests that the human subject harbors prostate cancer tumor that would garner a Gleason score of about 7.0 or above, if it is determined that the ratio falls in the range of about 5.9 to about 13.

### Embodiment 57

The method of Embodiment 56 which the human patient harbors a high grade prostate cancer.

### Embodiment 58

The method of any one of Embodiments 37-57 which suggests that the human subject harbors prostate cancer tumor that would garner a Gleason score of about 9.0 or above, if it is determined that the ratio falls in the range of about 15.5 to about 45.0.

### Embodiment 59

The method of any one of Embodiments 21-58 which suggests that the human subject harbors no disease if it is determined that the ratio is below 5.9.

### Embodiment 60

A method for confirming tumor metastasis to a pelvic lymph node of a prostate cancer patient, the method comprising:
administering to the patient an effective amount of a compound that selectively binds to prostate-specific membrane antigen (PSMA), the compound represented by Formula 1 or Formula 2 or a pharmaceutically acceptable salt thereof;
imaging a pelvis;
assessing a level of uptake of the compound by at least a portion of a pelvic lymph node of the prostate cancer patient compared to a level of uptake by a control tissue; and
confirming metastasis if it is determined that a ratio of the level of the compound by said at least a portion of the pelvic lymph node to the level of uptake by control tissue is at or above a predetermined threshold value;
wherein: Formula I and Formula 2 are:

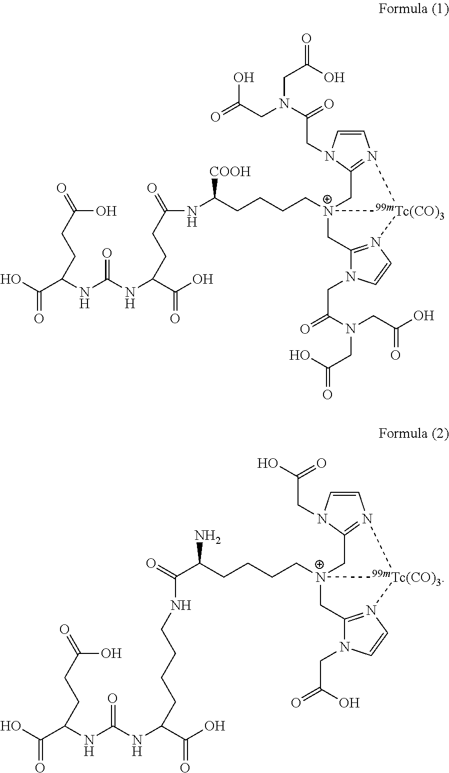

### Embodiment 61

The method of Embodiment 60, in which the predetermined threshold is chosen statistically to minimize undesirable effects of false positives and false negatives.

### Embodiment 62

The method of any one of Embodiments 60-61 in which the patient is administered an effective amount of a compound of Formula 1.

### Embodiment 63

The method of any one of Embodiments 60-62 in which the imaging is performed using a nuclear medicine tomographic imaging technique.

### Embodiment 64

The method of any one of Embodiments 60-63 in which the nuclear medicine tomographic imaging technique is selected from two-dimensional planar imaging, single-photon emission computed tomography (SPECT), or single-photon emission computed tomography combined with conventional computed tomography (SPECT/CT).

### Embodiment 65

The method of any one of Embodiments 60-64 in which the patient with confirmed metastasis to the pelvic lymph node is further subjected to radical prostatectomy in conjunction with radiation therapy, cryosurgery, anti-androgen therapy, chemotherapy or a combination of radiation therapy anti-androgen therapy and chemotherapy.

### Embodiment 66

The method of any one of embodiments 60-65 in which the control tissue is selected from normal prostate tissue, normal pelvic muscle or normal pelvic lymph node.

### Embodiment 67

The method of any one of Embodiments 60-66, wherein the pelvic lymph node has a mass of less than 6 mm in diameter.

### Embodiment 68

The method of any one of Embodiments 60-67, wherein the pelvic lymph node has a mass of less than 5 mm in diameter.

### Embodiment 69

The method of any one of Embodiments 60-68, wherein the pelvic lymph node has a mass of less than 3.5 mm in diameter.

### Embodiment 70

The method of any one of Embodiments 60-69, wherein the pelvic lymph node is detectable by SPECT/CT and has a mass of less than 3.5 mm in diameter.

### Embodiment 71

A method of monitoring a status of prostate cancer in a human subject, the method comprising:
administering to a human subject an effective amount of a gamma-emitting imaging agent comprising a prostate specific-membrane antigen (PSMA) recognition moiety and a radionuclide;
subjecting the human subject to a nuclear medicine tomographic imaging technique to obtain one or more images of at least a portion of prostate tissue that includes tumor lesions;
assessing a level of uptake of said gamma-emitting imaging agent by said at least a portion of prostate tissue compared to a level of uptake by control tissue;
determining a ratio of the level of uptake by said at least a portion of prostate tissue compared to the level of uptake by control tissue; and
comparing the ratio to a baseline ratio previously determined for the human subject.

### Embodiment 72

The method of Embodiment 71 in which the imaging agent is a glu-urea-glu or glu-urea-lys based imaging agent.

### Embodiment 73

The method of Embodiment 71 or 72 in which the imaging agent is one of:

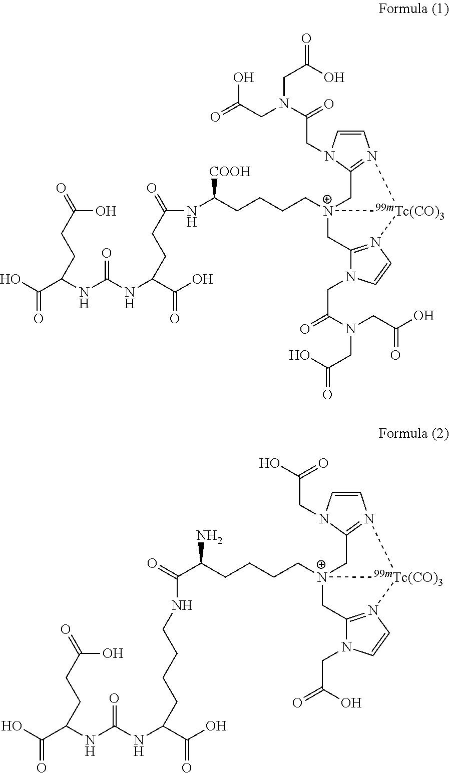

or a pharmaceutically acceptable salt thereof.

### Embodiment 74

The method of any one of Embodiments 71-73 in which the imaging step is carried out 1-6 hours after the administering step.

### Embodiment 75

The method of any one of Embodiments 71-74 which suggests a worsening of the prostate cancer if it is determined that the ratio is above the baseline ratio.

### Embodiment 76

The method of any one of Embodiments 71-75 which suggests that the prostate cancer has not worsened if it is determined that the ratio is at or below the baseline ratio.

### Embodiment 77

The method of any one of Embodiments 71-76 in which the patient is subjected to one or more prostate cancer treatment options if it is determined that the prostate cancer has worsened.

### Embodiment 78

A method of obtaining a SPECT/CT image of tissue expressing prostate-specific membrane antigen (PSMA) in vivo, the method comprising:
administering to a subject an effective amount of a Tc-99m chelate complex having an affinity for PSMA expressing tissue;
obtaining the SPECT/CT image of the subject in which the image provides clinical information sufficient to allow (i) staging of pathological disease comparable to a Gleason Score (GS) without a need for obtaining a biopsy, and (ii) minimization of false positive prostate cancer diagnosis compared to magnetic resonance imaging (MRI);
In which the affinity for PSMA expressing tissue is conveyed at least in part by either a Glu-Urea-Glu or Glu-Urea-Lys moiety on the Tc-99m chelate complex and the Tc-99m chelate complex includes a bis-imidazolylmethylamine group complexed to the Tc-99m.

### Embodiment 79

The method of Embodiment 78 which provides a degree of specificity and sensitivity for detection of primary or metastasized prostate cancer that is greater than MRI detection or conventional bone scan detection,

### Embodiment 80

The method of Embodiment 78 or 79 further comprising evaluating the image by assigning a background region and a prostate region, a seminal vesicle, or both a prostate region and a seminal vesicle a Lesion Visualization Grading Score of from 0 to 4, with 0 indicating equivalence to the background activity and no lesions observed and 4 indicating greater than all other activity.

### Embodiment 81

The method of any one of Embodiments 78-80, wherein a positive score is observed in a subject having a target to background ratio of greater than 4:1, and the background region is observed from normal tissue within the pelvis.

### Embodiment 82

The method of any one of Embodiments 78-81, wherein the target to background ratio is greater than 5:1.

### Embodiment 83

The method of any one of Embodiments 78-82, wherein the target to background ratio is greater than 6:1.

### Embodiment 84

The method of any one of Embodiments 78-83, wherein the Tc-99m chelate complex is:

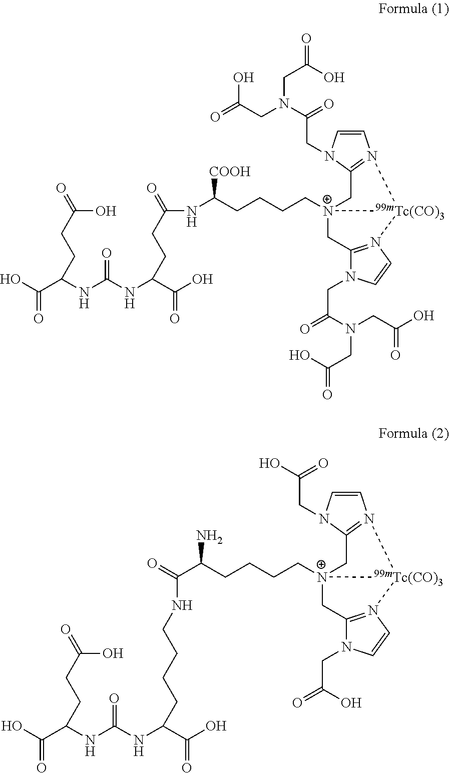

or a pharmaceutically acceptable salt thereof

### Embodiment 85

The method of any one of Embodiments 78-84 in which the observing step is carried out 1-6 hours after the administering step.

### Embodiment 86

The method of any one of Embodiments 78-85, wherein the method is capable of correctly characterizing prostate cancer in greater than 90% of patients compared to magnetic resonance imaging which is capable of correctly characterizing prostate cancer in 81% of patients.

### Embodiment 87

A method for detecting tumor metastasis to at least a portion of a bone or a soft tissue of a prostate cancer patient, the method comprising:
administering to the patient an effective amount of a gamma-emitting transition metal complex conjugated to a targeting moiety that selectively binds to prostate-specific membrane antigen (PSMA) in at least the portion of the bone or soft tissue;
imaging a region of bone or soft in the patient;
assessing a level of uptake of said gamma-emitting transition metal complex by the bone tissue compared to a level of uptake by a control bone or soft tissue; and
confirming tumor metastasis if it is determined that a ratio of the level of the gamma-emitting transition metal complex uptake by the portion of the bone tissue to the level of uptake by control bone tissue is at or above a predetermined threshold value.

### Embodiment 88

The method of Embodiment 87, wherein the soft tissue is lung tissue.

### Embodiment 89

The method of Embodiment 87 or 88 in which the patient is administered an effective amount of a compound of Formula 1 or Formula II:

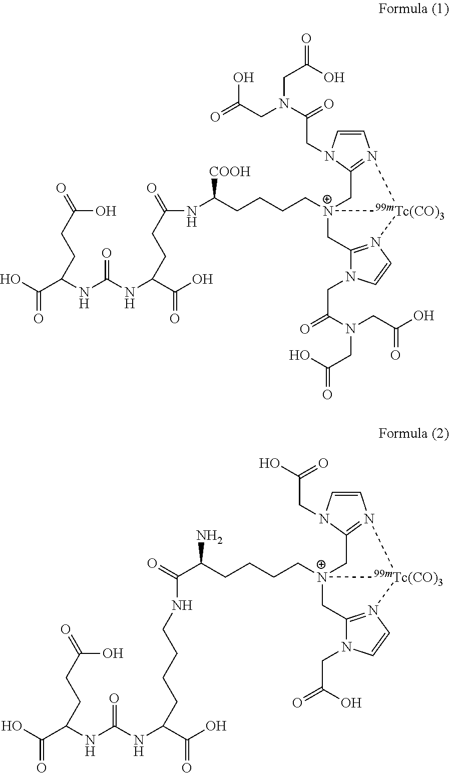

or a pharmaceutically acceptable salt thereof

### Embodiment 90

The method of Embodiment 86, 87, 88, or 89 in which the imaging is performed using a nuclear medicine tomographic imaging technique.

### Embodiment 91

The method of Embodiment 90 in which the nuclear medicine tomographic imaging technique is selected from two-dimensional planar imaging, single-photon emission computed tomography (SPECT), or single-photon emission computed tomography combined with conventional computed tomography (SPECT/CT).

### Embodiment 92

A method of identifying prostate tumor metastasis to a lymph node, the method comprising:
administering to a subject suspected of having prostate cancer an effective amount of a compound represented by Formula 1 or Formula 2 or a pharmaceutically acceptable salt thereof;
imaging the subject using a nuclear medicine tomographic imaging technique; and
confirming a mass in the lymph node of the subject;
wherein: Formula 1 and Formula 2 are:

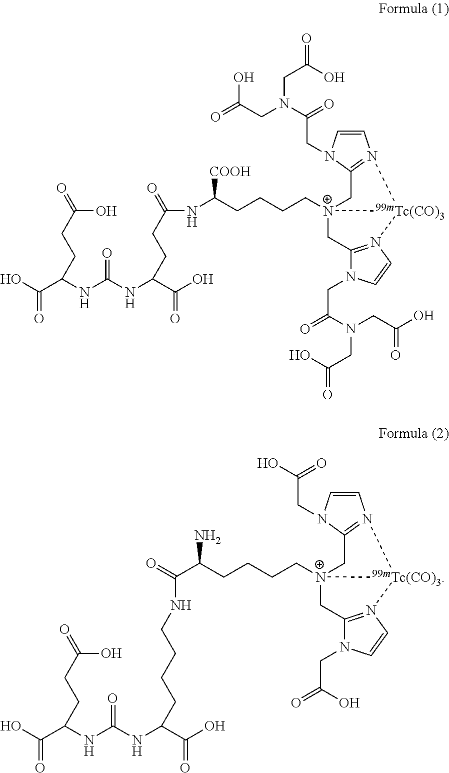

### Embodiment 93

The method of Embodiment 92, wherein the mass is at least about 2 mm in diameter.

### Embodiment 94

The method of Embodiment 92 or 93, wherein the mass is from about 2 mm to about 10 mm in diameter.

### Embodiment 95

The method of Embodiment 92, 93, or 94 in which the nuclear medicine tomographic imaging technique is selected from two-dimensional planar imaging, single-photon emission computed tomography (SPECT), or single-photon emission computed tomography combined with conventional computed tomography (SPECT/CT).

### Embodiment 96

The method of Embodiment 95, wherein the pelvic lymph node is detectable by SPECT/CT and has a mass of less than 3.5 mm in diameter.

### Embodiment 97

The method of any one of Embodiments 92-96, wherein the effective amount is about 20 mCi.

### Embodiment 98

The method of any one of Embodiments 92-97, wherein the lymph node is a pelvic lymph node.

### Embodiment 99

A method of monitoring the efficacy of prostate cancer treatment, the method:
administering to a subject prior to undergoing treatment for prostate cancer a first amount of a compound represented by Formula 1 or Formula 2 or a pharmaceutically acceptable salt thereof;
   treating the subject for prostate cancer;
   administering to a subject undergoing, or having undergone, treatment for prostate cancer a second amount of a compound represented by Formula 1 or Formula 2 or a pharmaceutically acceptable salt thereof;
   imaging the subject using a nuclear medicine tomographic imaging technique; and
   confirming that expression of prostate specific membrane antigen is reduced in the subject after treatment;
   wherein: Formula 1 and Formula 2 are:

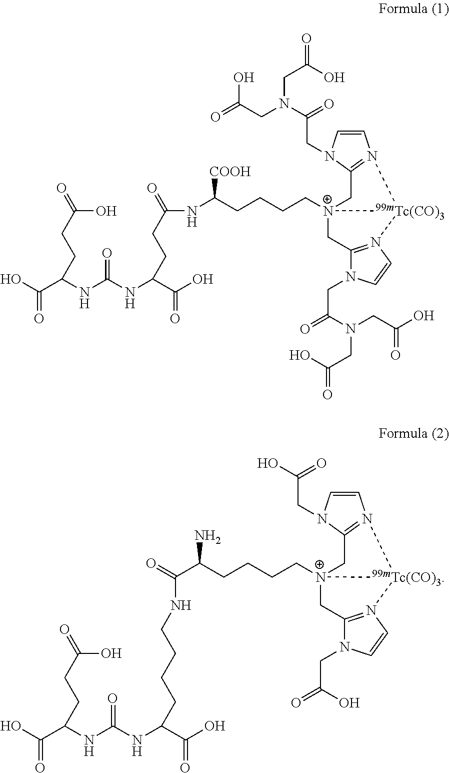

### Embodiment 100

The method of Embodiment 99, wherein the treating is conducted with hormonal therapy, antimitotic chemotherapy, PSMA antibody-drug conjugate, or a combination of any two or more thereof.

### Embodiment 101

A method of monitoring or assessing a status of prostate cancer in a human subject, the method comprising:
determining a level of uptake of a gamma-emitting imaging agent comprising a prostate specific-membrane antigen (PSMA) recognition moiety and a radionuclide by at least a portion of prostate tissue of a human subject, which includes one or more tumor lesions;
determining a ratio of (a) the level of uptake of said gamma-emitting imaging agent by said at least a portion of prostate tissue, and (b) a level of uptake of said gamma-emitting imaging agent by a control tissue of said human subject; and
comparing said ratio to a baseline ratio previously determined for said human subject.

### Embodiment 102

The method of Embodiment 101 in which said ratio, if found to be higher than said baseline ratio, is indicative of disease progression.

### Embodiment 103

The method of Embodiment 101 or 102 in which said ratio, if found to be lower than said baseline ratio, is indicative of disease remission,

### Embodiment 104

A non-invasive method of assessing a degree of disease aggressiveness in a human subject diagnosed with prostate cancer, the method comprising recording a level of uptake of a radiolabelled MIP-1404 or MIP-1405 by diseased tissue of a human subject diagnosed with prostate cancer and determining from said level of uptake a degree of disease aggressiveness in said human subject.

### Embodiment 105

The method of Embodiment 104 in which said determination involves calculating a ratio of (a) the level of uptake of said radiolabelled MIP-1404 or MIP-1405 by said diseased tissue, and (b) a level of uptake of said ^{99m}Tc-MIP-1404 or ^{99m}Tc-MIP-1405 by a control tissue of said human subject.

### Embodiment 106

The method of Embodiment 104 or 105 which further comprises comparing the calculated ratio with a predetermined threshold.

### Embodiment 107

The method of Embodiment 106 in which the predetermined threshold is from about 25 to about 40.

### Embodiment 108

The method of any one of Embodiments 104-107, wherein the radiolabelled MIP-1404 is ^{99m}Tc-trofolastat chloride and the radiolabelled MIP-1405 is ^{99m}Tc-MIP-1405.

### Embodiment 109

An in vivo method of assessing a likelihood of a presence of metastatic disease in a human subject diagnosed with prostate cancer, the method comprising recording a level of uptake of a radiolabelled MIP-1404 or MIP-1405 by diseased tissue, which includes a primary tumor, of a human subject diagnosed with prostate cancer and determining from said level of uptake a likelihood of a presence of metastatic disease in said human subject.

### Embodiment 110

The method of Embodiment 109 in which said determination involves calculating a ratio of (a) the level of uptake of said radiolabelled MIP-1404 or MIP-1405 by said diseased tissue, and (b) a level of uptake of said radiolabelled MIP-1404 or MIP-1405 by a control tissue of said human subject.

### Embodiment 111

The method of Embodiment 110 which further comprises comparing the calculated ratio with a predetermined threshold.

### Embodiment 112

The method of Embodiment 111 in which the predetermined threshold is at least about 30.

### Embodiment 113

The method of Embodiment 109 or 110, wherein the radiolabelled MIP-1404 is ^{99m}Tc-trofolastat chloride and the radiolabelled MIP-1405 is ^{99m}Tc-MIP-1405.

### Embodiment 114

The method of any one of Embodiments 109-113, wherein the human subject has not received prostate cancer treatment prior to the method.

### Embodiment 115

A non-surgical method of diagnosing metastatic disease in a patient clinically diagnosed as having prostate cancer, which method does not rely on histopathology of a prostate or a lymph node, the method comprising:
administering to the patient an effective amount of a compound that selectively binds to prostate-specific membrane antigen (PSMA), the compound represented by Formula 1 or Formula 2 or a pharmaceutically acceptable salt thereof;
determining a level of uptake of the compound in the prostate of the patient as a tumor (T) level;
determining a level of uptake of the compound in a control tissue as a baseline (B) level; and
confirming lymph node involvement if a ratio of T:B is at, or above, a predetermined threshold value;
wherein: Formula I and Formula 2 are:

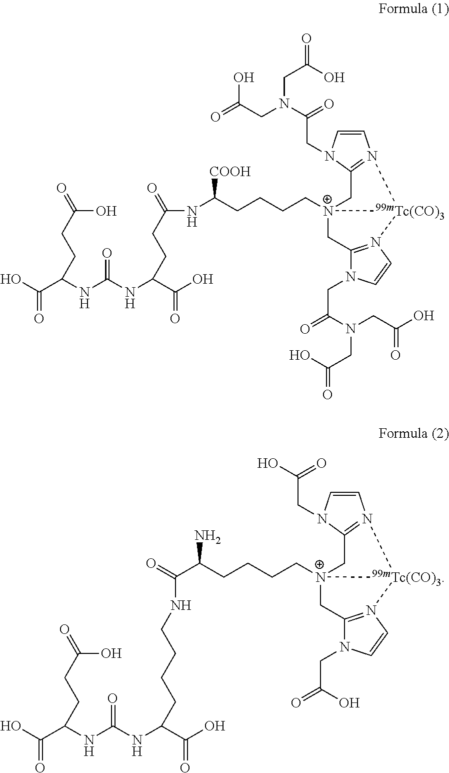

### Embodiment 116

The method of Embodiment 115, wherein the clinical diagnosis of prostate cancer is determined using a PSA value, digital rectal examination, trans-rectal ultra sound, symptomology, or a combination of any two or more thereof

### Embodiment 117

The method of Embodiment 115 or 116, wherein the predetermined threshold is about 30.

### Embodiment 118

The method of Embodiment 115, 116, or 117, wherein the T:B ratio is ≧30, indicating a diagnosis of metastatic disease.

### Embodiment 119

The method of any one of Embodiments 115-118, wherein the T:B ratio is ≦30, indicating a diagnosis of negative metastatic disease.

### Embodiment 120

The method of any one of Embodiments 115-119, wherein the patient has not received a prior prostate cancer treatment.

### Embodiment 121

The method of any one of Embodiments 115-120, wherein the determining comprises obtaining an image of the patient using nuclear medicine tomographic imaging techniques.

### Embodiment 122

The method of any one of Embodiments 115-121, wherein the compound is ^{99m}Tc-trofolastat chloride.

### Embodiment 123

The method of any one of Embodiments 115-122 having a sensitivity of about 90%.

### Embodiment 124

The method of any one of Embodiments 115-122, wherein the T:B ratio correlates with a Gleason score.

### Embodiment 125

A non-surgical method of identifying a severity level of prostate cancer in a patient harboring biopsy-confirmed prostate cancer, the method comprising:
administering to the patient an effective amount of a compound that is ^{99m}Tc-trofolastat chloride;
determining a level of uptake of the compound in the prostate of the patient as a tumor (T) level;
determining a level of uptake of the compound in a control tissue as a baseline (B) level; and
assigning a severity level in terms of Gleason score if a ratio of T:B is at, or above, a predetermined threshold value.

### Embodiment 126

The method of Embodiment 125, wherein when the threshold value of >5.9 corresponds to a Gleason score of about 7.0 or greater.

### Embodiment 127

The method of Embodiment 125, wherein when the threshold value of about 15.5 or greater corresponds to a Gleason score of about 9.0 or greater.

### Embodiment 128

The method of Embodiment 125, 126, or 127, wherein the patient has not received a prior prostate cancer treatment.

### Embodiment 129

The method of Embodiment 125, 126, 127, or 128, wherein the determining comprises obtaining an image of the patient using nuclear medicine tomographic imaging techniques.

### Embodiment 130

A method of assigning a level of cancer severity of a patient diagnosed with prostate cancer, the method comprising:
determining a level of uptake of a compound that is ^{99m}Tc-trofolastat chloride by prostate tissue of a patient diagnosed with prostate cancer (a target T level);
determining a level of uptake of the compound by a control tissue of the prostate cancer patient (a baseline B level); and
assigning a level of cancer severity of the patient based on a ratio of the target T level to the baseline B level (T:B).

### Embodiment 131

A method for confirming lymph node involvement in a metastatic prostate cancer of a patient, the method comprising:
administering to the patient an effective amount of a compound represented by Formula 1 or Formula 2 or a pharmaceutically acceptable salt thereof;
determining a level of uptake of the compound by the prostate of the patient as a target (T) level;
determining a level of uptake of the compound by control tissue of the patient as a baseline (B) level; and
confirming lymph node involvement if a ratio of T:B is at, or above, a predetermined threshold value;
wherein: Formula I and Formula 2 are:

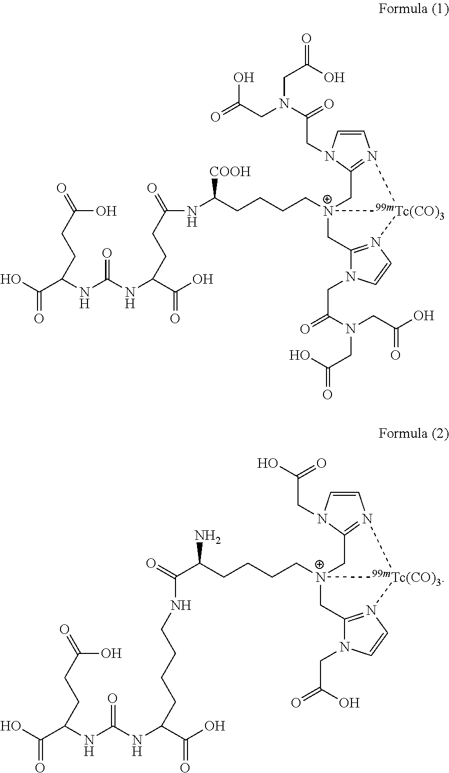

### Embodiment 132

A kit comprising a radioactive diagnostic agent for nuclear medicine tomographic imaging of the prostate and instructions for diagnosing clinically-significant prostate cancer based upon a quantitative score (T:B ratio).

### Embodiment 133

A method of obtaining a SPECT/CT image of tissue expressing prostate-specific membrane antigen (PSMA) in vivo, the method comprising:
administering to a subject an effective amount of a Tc-99m chelate complex having an affinity for PSMA expressing tissue;
obtaining the SPECT/CT image of the subject in which the image provides clinical information sufficient to allow (i) staging of pathological disease comparable to a Gleason Score (GS) without a need for obtaining a biopsy, and (ii) minimization of false positive prostate cancer diagnosis compared to magnetic resonance imaging (MRI);
in which the affinity for PSMA expressing tissue is conveyed at least in part by either a Glu-Urea-Glu moiety or a Glu-Urea-Lys moiety of the Tc-99m chelate complex, and the chelate includes a bis-imidazolylmethylamine group.

### Embodiment 134

A method for detecting tumor metastasis to at least a portion of a bone or a soft tissue of a prostate cancer patient, the method comprising:
administering to the patient an effective amount of a gamma-emitting transition metal complex conjugated to a targeting moiety that selectively binds to prostate-specific membrane antigen (PSMA) in at least the portion of the bone or soft tissue;
imaging a region of the patient, including the at least the portion of the bone or soft tissue; assessing a level of uptake of said gamma-emitting transition metal complex by the at least the portion of the bone or soft tissue compared to a level of uptake by a control bone or soft tissue; and
confirming tumor metastasis if it is determined that a ratio of the level of uptake by the at least the portion of the bone or soft tissue to the level of uptake by the control bone or soft tissue is at or above a predetermined threshold value.

### Embodiment 135

A method of monitoring the efficacy of prostate cancer treatment, the method:
administering to a subject prior to undergoing treatment for prostate cancer a first amount of a compound represented by Formula 1 or Formula 2 or a pharmaceutically acceptable salt thereof and obtaining an initial image using a nuclear medicine tomographic imaging technique;
treating the subject for prostate cancer;
administering to a subject undergoing, or having undergone, treatment for prostate cancer a second amount of a compound represented by Formula 1 or Formula 2 or a pharmaceutically acceptable salt thereof and obtaining a subsequent image using the nuclear medicine tomographic imaging technique; and
confirming that expression of prostate specific membrane antigen is reduced in the subject undergoing, or having undergone, treatment;
wherein: Formula 1 and Formula 2 are:

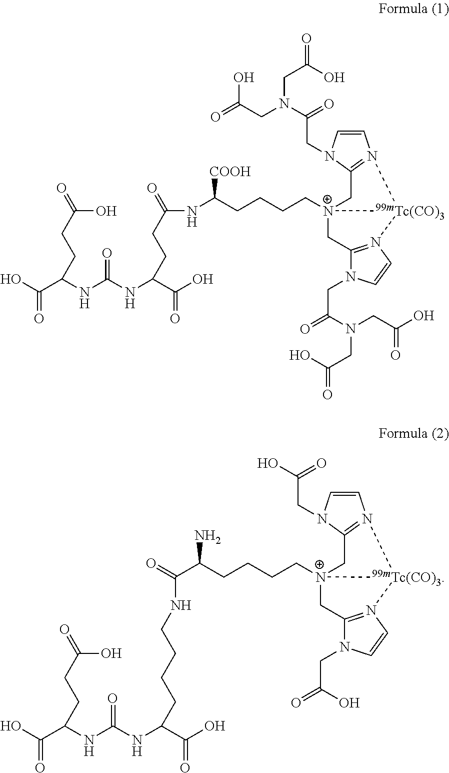

### Embodiment 136

A non-surgical method of diagnosing metastatic disease in a patient clinically diagnosed as having prostate cancer, which method does not rely on histopathology of a prostate or a lymph node, the method comprising:
administering to the patient an effective amount of a compound represented by Formula 1 or Formula 2 or a pharmaceutically acceptable salt thereof;
determining a level of uptake of the compound by the prostate of the patient as a tumor (T) level;
determining a level of uptake of the compound by a control tissue as a baseline (B) level; and confirming metastatic disease if a ratio of T:B is at, or above, a predetermined threshold value;
wherein: Formula I and Formula 2 are:

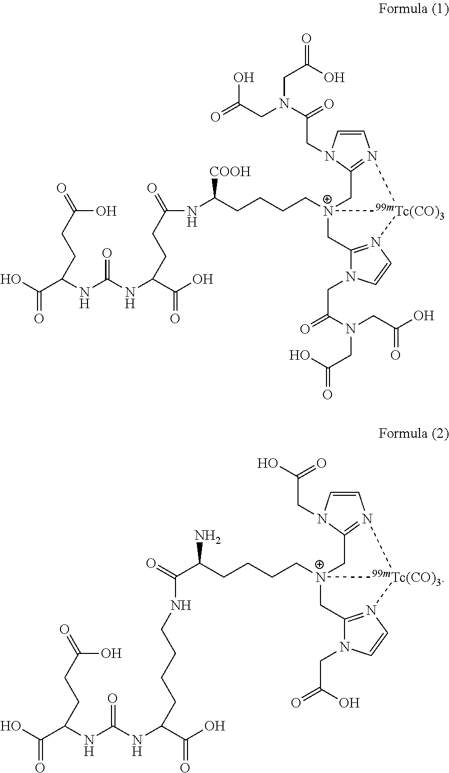

### Embodiment 137

A non-surgical method of identifying a severity level of prostate cancer in a patient harboring biopsy-confirmed prostate cancer, the method comprising:
administering to the patient an effective amount of a compound that is ^{99m}Tc-trofolastat chloride;
determining a level of uptake of the compound in the prostate of the patient as a tumor (T) level;
determining a level of uptake of the compound in a control tissue as a baseline (B) level; and assigning a severity level based on a ratio of T:B.

### Equivalents

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

What is claimed is:

1. A method of assigning a level of cancer severity of a patient diagnosed with prostate cancer, the method comprising:
determining a level of uptake of a compound that is ^{99m}Tc-trofolastat chloride by prostate tissue of a patient diagnosed with prostate cancer (a target T level);
determining a level of uptake of the compound by a control tissue of the prostate cancer patient (a baseline B level); and
assigning a level of cancer severity of the patient based on a ratio of the target T level to the baseline B level (T:B).

2. The method of claim 1, wherein the method is a non-surgical method.

3. The method of claim 1, wherein a T:B ratio of ≦5.9 identifies the patient without clinically-significant prostate cancer at the time of the image acquisition.

4. The method of claim 1, wherein when a T:B ratio greater than about 5.9 is highly sensitive for identifying the patient with clinically significant prostate cancer at the time of the image acquisition.

5. The method of claim 1, wherein a T:B ratio of >15 is highly specific for identifying the patient with clinically significant prostate cancer at the time of image acquisition.

6. The method of claim 1, wherein the determining comprises obtaining the image of the patient using nuclear medicine tomographic imaging techniques.

7. The method of claim 1, wherein the patient has not received a prior prostate cancer treatment.

8. A method for confirming tumor metastasis in a prostate cancer patient, the method comprising:
administering to a patient diagnosed with prostate cancer an effective amount of a compound, the compound represented by Formula 1 or Formula 2 or a pharmaceutically acceptable salt thereof;
obtaining a level of uptake of the compound by prostate tissue of the prostate cancer patient (a target T level);
obtaining a level of uptake of the compound by a control tissue of the prostate cancer patient (a baseline B level); and
confirming metastasis if a T:B ratio is at, or above, a predetermined threshold value;
wherein: Formula I and Formula 2 are:

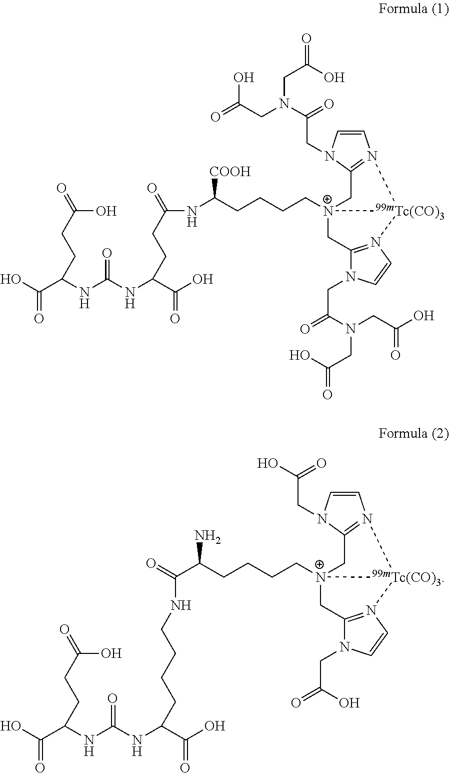

9. The method of claim 8, in which the predetermined threshold is chosen statistically to minimize undesirable effects of false positives and false negatives.

10. The method of claim 8, wherein the predetermined threshold is about 30.

11. The method of claim 8 in which the control tissue is normal prostate tissue, normal pelvic muscle, or normal pelvic lymph node.

12. A kit comprising a first container including a free ligand MIP-1404, a second container including a ^{99m}Tc radionuclide, and instructions for producing ^{99m}Tc-trofolastat for: identifying a severity level of prostate cancer in a patient, confirming lymph node involvement in metastatic prostate cancer, confirming tumor metastasis, monitoring a status of prostate cancer, obtaining a SPECT/CT image of tissue expressing prostate-specific membrane antigen (PSMA) in vivo, detecting tumor metastasis to at least a portion of a bone or a soft tissue of a prostate cancer patient, identifying prostate tumor metastasis to a lymph node, monitoring the efficacy of prostate cancer treatment, monitoring or assessing a status of prostate cancer in a human subject, a non-invasive method of assessing a degree of disease aggressiveness in a human subject diagnosed with prostate cancer, assessing a likelihood of a presence of metastatic disease in a human subject diagnosed with prostate cancer, diagnosing metastatic disease in a patient clinically diagnosed as having prostate cancer, or identifying a severity level of prostate cancer in a patient harboring biopsy-confirmed prostate cancer.

13. A kit comprising a radioactive diagnostic agent for nuclear medicine tomographic imaging of the prostate and instructions for diagnosing clinically-significant prostate cancer based upon a quantitative score (T:B ratio).

14. The kit of claim 13, wherein the instructions provide that a T:B threshold value ≦5.9 is indicative of clinically non-significant prostate cancer.

15. The kit of claim 13, wherein the instructions provide that a T:B threshold value >5.9 is indicative of clinically significant prostate cancer.

16. The kit of claim 13, wherein the instructions provide that a T:B threshold value >15 is indicative of clinically significant prostate cancer

17. The kit of claim 13, wherein the instructions provide that a T:B threshold value >30 is indicative of metastatic disease.

18. A method of monitoring a status of prostate cancer in a human subject, the method comprising:
administering to a human subject an effective amount of a gamma-emitting imaging agent comprising a prostate specific-membrane antigen (PSMA) recognition moiety and a radionuclide;
subjecting the human subject to a nuclear medicine tomographic imaging technique to obtain one or more images of at least a portion of prostate tissue that includes tumor lesions;
assessing a level of uptake of said gamma-emitting imaging agent by said at least a portion of prostate tissue compared to a level of uptake by control tissue;
determining a ratio of the level of uptake by said at least a portion of prostate tissue to the level of uptake by control tissue; and
comparing the ratio to a baseline ratio previously determined for the human subject.

19. The method of claim 18 in which the imaging agent is a glu-urea-glu or glu-urea-lys based imaging agent.

20. The method of claim 18 in which the imaging agent is one of: 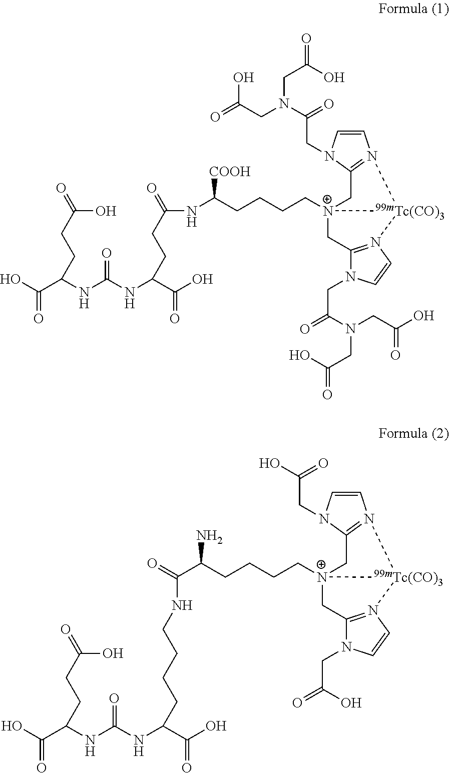
or a pharmaceutically acceptable salt thereof.

21. A method of obtaining a SPECT/CT image of tissue expressing prostate-specific membrane antigen (PSMA) in vivo, the method comprising:
administering to a subject an effective amount of a Tc-99m chelate complex having an affinity for PSMA expressing tissue;
obtaining the SPECT/CT image of the subject in which the image provides clinical information sufficient to allow (i) staging of pathological disease comparable to a Gleason Score (GS) without a need for obtaining a biopsy, and (ii) minimization of false positive prostate cancer diagnosis compared to magnetic resonance imaging (MRI);
in which the affinity for PSMA expressing tissue is conveyed at least in part by either a Glu-Urea-Glu moiety or a Glu-Urea-Lys moiety of the Tc-99m chelate complex, and the chelate includes a bis-imidazolylmethylamine group.

22. The method of claim 21 which provides a degree of specificity and sensitivity for detection of primary or metastasized prostate cancer that is greater than MRI detection or conventional bone scan detection,

23. A method for detecting tumor metastasis to at least a portion of a bone or a soft tissue of a prostate cancer patient, the method comprising:
administering to the patient an effective amount of a gamma-emitting transition metal complex conjugated to a targeting moiety that selectively binds to prostate-specific membrane antigen (PSMA) in at least the portion of the bone or soft tissue;
imaging a region of the patient, including the at least the portion of the bone or soft tissue;
assessing a level of uptake of said gamma-emitting transition metal complex by the at least the portion of the bone or soft tissue compared to a level of uptake by a control bone or soft tissue; and
confirming tumor metastasis if it is determined that a ratio of the level of uptake by the at least the portion of the bone or soft tissue to the level of uptake by the control bone or soft tissue is at or above a predetermined threshold value.

24. A method of monitoring or assessing a status of prostate cancer in a human subject, the method comprising:
determining a level of uptake of a gamma-emitting imaging agent comprising a prostate specific-membrane antigen (PSMA) recognition moiety and a radionuclide by at least a portion of prostate tissue of a human subject, which includes one or more tumor lesions;
determining a ratio of (a) the level of uptake of said gamma-emitting imaging agent by said at least a portion of prostate tissue, and (b) a level of uptake of said gamma-emitting imaging agent by a control tissue of said human subject; and
comparing said ratio to a baseline ratio previously determined for said human subject.

25. A non-invasive method of assessing a degree of disease aggressiveness in a human subject diagnosed with prostate cancer, the method comprising recording a level of uptake of a compound represented by Formula 1 or Formula 2 or a pharmaceutically acceptable salt thereof by diseased tissue of a human subject diagnosed with prostate cancer and determining from said level of uptake a degree of disease aggressiveness in said human subject, wherein: Formula 1 and Formula 2 are:

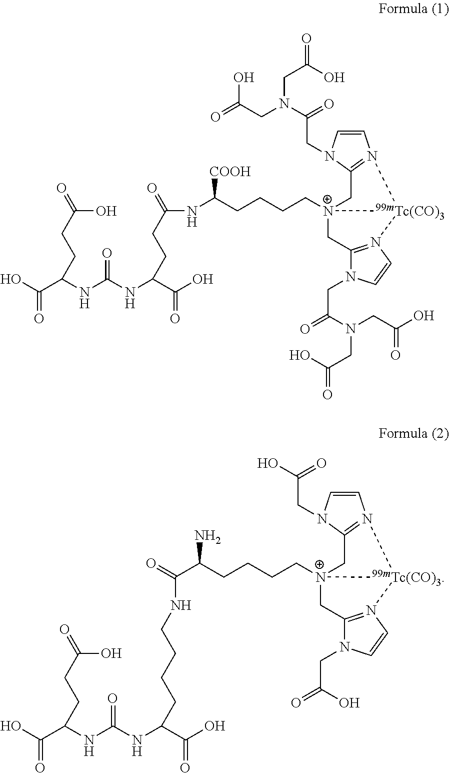

26. An in vivo method of assessing a likelihood of a presence of metastatic disease in a human subject diagnosed with prostate cancer, the method comprising recording a level of uptake of a compound represented by Formula 1 or Formula 2 or a pharmaceutically acceptable salt thereof by diseased tissue, which includes a primary tumor, of a human subject diagnosed with prostate cancer and determining from said level of uptake a likelihood of a presence of metastatic disease in said human subject, wherein: Formula 1 and Formula 2 are: [chemical structure]
